(19) United States
(12) Patent Application Publication
(10) Pub. No.: US 2018/0244786 A1
(43) Pub. Date: Aug. 30, 2018

(54) ANTI IL-3R ALPHA AGENTS AND USES THEREOF

(71) Applicant: CSL Limited, Parkville (AU)

(72) Inventors: Angel Francisco LOPEZ, Medindie (AU); Timothy Robert HERCUS, Clapham (AU); Sophie Elizabeth BROUGHTON, Fitzroy (AU); Michael William PARKER, Newport (AU); Nicholas WILSON, Parkville (AU); Matthew HARDY, Parkville (AU)

(21) Appl. No.: 15/724,145

(22) Filed: Oct. 3, 2017

Related U.S. Application Data
(63) Continuation of application No. 14/775,631, filed on Sep. 11, 2015, now abandoned.
(60) Provisional application No. 61/783,289, filed on Mar. 14, 2013.

(51) Int. Cl. C07K 16/28

(52) U.S. Cl. CPC C07K 16/2866; C07K 2317/565; C07K 2317/56; C07K 2317/76; C07K 2317/73; C07K 2317/34

(57) ABSTRACT

The present disclosure provides antibodies and antigen binding fragments thereof that bind to an epitope within an interleukin-3 receptor α.

### RELATED APPLICATION

The present application is a continuation of U.S. patent application Ser. No. 14/775,631, entitled “Anti IL-3R alpha agents and uses thereof” filed on Sep. 11, 2015, which application is a national stage entry of International Application No. PCT/AU2014/000265, entitled “Anti IL-3R alpha agents and uses thereof” filed on Mar. 13, 2014, which claims priority from U.S. Patent Application No. 61/783,289, entitled “Anti IL-3R alpha agents and uses thereof” filed on Mar. 14, 2013. The entire contents of these applications are hereby incorporated by reference.

### SEQUENCE LISTING

The Sequence Listing associated with this application is provided in text format in lieu of a paper copy, and is hereby incorporated by reference into the specification. The name of the text file containing the Sequence Listing is RICE_003_02US_ST25.txt. The text file is 18 KB, was created on Oct. 3, 2017, and is being submitted electronically via EFS-Web.

### FIELD

The present disclosure relates to compounds that bind to interleukin 3 receptor alpha (IL-3Rα) and uses thereof.

### BACKGROUND

The functional interleukin 3 receptor is a heterodimer that comprises a specific alpha chain (IL-3Rα/CD123) and a “common” IL-3 receptor beta chain (β_{c}; CD131) that is shared with the receptors for granulocyte macrophage colony stimulating factor (GM-CSF) and interleukin 5 (IL-5).

IL-3Rα is a type I transmembrane protein with a deduced Molecular Weight of about 41 kDa containing an extracellular domain involved in IL-3 binding, a transmembrane domain and a short cytoplasmic tail of about 50 amino acids. The extracellular domain is composed of two regions: an N-terminal region of about 100 amino acids, the sequence of which exhibits similarity to equivalent regions of the GM-CSF and IL-5 receptor alpha-chains; and a region proximal to the transmembrane domain that contains four conserved cysteine residues and a WSXWS motif, common to other members of this cytokine receptor family.

The IL-3 binding domain comprises about 200 amino acid residue cytokine receptor motifs (CRMs) made up of two Ig-like folding domains. The extracellular domain of IL-3Rα is highly glycosylated, with N-glycosylation necessary for both ligand binding and receptor signaling.

IL-3Rα is expressed widely throughout the hematopoietic system including hematopoietic progenitors, mast cells, erythroid cells, megakaryocytes, basophils, eosinophils, monocytes/macrophages, neutrophils, plasmacytoid dendritic cells (pDCs) and CD5⁺ B-lymphocytes. Non-hematopoietic cells such as Leydig cells, endothelial cells and stromal cells also express IL-3Rα.

IL-3Rα is also expressed by cells involved in certain disease states including myelodysplastic syndrome, myeloid leukemia (for example, acute myelogenous leukemia (AML)), malignant lymphoproliferative disorders such as lymphoma, allergies and autoimmune disease, such as lupus, Sjögren's syndrome or scleroderma. Accordingly, compounds that bind to IL-3Rα are desirable for therapeutic applications.

### SUMMARY

The present disclosure is based on the inventors' identification of an epitope within IL-3Rα bound by compounds that neutralize signaling by IL-3, e.g., neutralizing antibodies.

The inventors have refined the region to which such compounds bind, e.g., to an epitope within amino acids 51-100 of SEQ ID NO: 1. Thus, the present disclosure provides a compound (such as, an antibody or antigen binding fragment thereof) that specifically binds to an epitope within IL-3Rα and is capable of neutralizing IL-3 signaling, wherein the epitope is in a region corresponding to amino acids 51-100 of SEQ ID NO: 1.

In one example, the present disclosure provides a compound (e.g., an antibody or antigen binding fragment thereof) which specifically binds to an epitope within an IL-3Rα chain, the epitope comprising one or more of amino acids 84 and/or 89 of SEQ ID NO: 1.

In one example, the present disclosure provides a compound (e.g., an antibody or antigen binding fragment thereof) which specifically binds to an epitope within an interleukin (IL)-3Rα chain, the epitope comprising residues positioned within a region comprising amino acids 58, 59 and 61 of SEQ ID NO: 1.

In one example, the present disclosure provides a compound (e.g., an antibody or antigen binding fragment thereof) which specifically binds to an epitope within an IL-3Rα chain, the epitope comprising amino acid 51 of SEQ ID NO: 1.

In one example, the present disclosure provides a compound (e.g., an antibody or antigen binding fragment thereof) which specifically binds to an epitope within an IL-3Rα chain, the epitope comprising one or more residues selected from the group consisting of amino acids 51, 58, 59, 61, 84, 85 and 89 of SEQ ID NO: 1.

In one example, the epitope comprises the following:

(i) amino acid 51 of SEQ ID NO: 1;
(ii) one or more of amino acids 58, 59 and/or 61 of SEQ ID NO: 1; and
(iii) one or more of amino acids 84 89 of SEQ ID NO: 1.

In one example, the epitope comprises:

(i) amino acid 51 of SEQ ID NO: 1;
(ii) one or more residues selected individually or collectively from 58, 59 and 61 of SEQ ID NO: 1; and
(iii) one or more residues selected individually or collectively from 84 and/or 89 of SEQ ID NO: 1.

The present disclosure also provides a compound (such as an antibody or antigen binding fragment thereof) which specifically binds to an epitope within an IL-3Rα chain, the epitope comprising residues corresponding to at least one of amino acids 51 or 59 of SEQ ID NO: 1.

In one example, the epitope comprises residues corresponding to amino acids 51 and 59 of SEQ ID NO: 1.

In one example, the epitope additionally comprises a residue corresponding to amino acid 58 of SEQ ID NO: 1.

In one example, the epitope additionally comprises a residue corresponding to amino acid 61 of SEQ ID NO: 1.

In one example, the epitope additionally comprises a residue corresponding to amino acid 84 of SEQ ID NO: 1.

In one example, the epitope additionally comprises a residue corresponding to amino acid 89 of SEQ ID NO: 1.

In one example, the present disclosure provides a compound (e.g., an antibody or antigen binding fragment thereof) which specifically binds to an epitope within an IL-3Rα chain comprising one or more (or all) residues corresponding to amino acids 51, 58, 59, 61, 84 and/or 89 of SEQ ID NO: 1.

In one example the epitope comprises residues corresponding to amino acids 51, 58, 59, 61, 84 and 89 of SEQ ID NO: 1.

The present disclosure also provides a compound (such as, an antibody or antigen binding fragment thereof) which specifically binds to an IL-3Rα chain, wherein the level of binding of the compound to a polypeptide comprising a sequence set forth in SEQ ID NO: 1 comprising one or more (or all) of the following amino acid substitutions:

(i) glutamic acid at position 51 is substituted with alanine or glutamine;
(ii) tyrosine at position 58 is substituted with alanine or phenylanine;
(iii) serine at position 59 is substituted with alanine;
(iv) proline at position 61 is substituted with alanine or threonine;
(v) arginine at position 84 is substituted with alanine or lysine; and/or
(vi) proline at position 89 is substituted with threonine,

is lower than the level of binding of the protein to a polypeptide of SEQ ID NO: 1.

The present disclosure also provides a compound (such as, an antibody or antigen binding fragment thereof) which specifically binds to an IL-3Rα chain, wherein the level of binding of the compound to a polypeptide comprising a sequence set forth in SEQ ID NO: 1 comprising one or more (or all) of the following combinations of amino acid substitutions:

(i) glutamic acid at position 51 is substituted with glutamine and tyrosine at position 58 is substituted with phenylanine;
(ii) glutamic acid at position 51 is substituted with glutamine and serine at position 59 is substituted with alanine;
(iii) glutamic acid at position 51 is substituted with glutamine and proline at position 61 is substituted with threonine;
(iv) glutamic acid at position 51 is substituted with glutamine and arginine at position 84 is substituted with lysine;
(v) glutamic acid at position 51 is substituted with glutamine and proline at position 89 is substituted with threonine;
(vi) tyrosine at position 58 is substituted with phenylanine and serine at position 59 is substituted with alanine;
(vii) serine at position 59 is substituted with alanine and proline at position 61 is substituted with threonine
(viii) serine at position 59 is substituted with alanine and arginine at position 84 is substituted with lysine;
(ix) serine at position 59 is substituted with alanine and proline at position 89 is substituted with threonine;
(x) proline at position 61 is substituted with threonine and arginine at position 84 is substituted with lysine; and/or
(xi) proline at position 61 is substituted with threonine and proline at position 89 is substituted with threonine,

is lower than the level of binding of the protein to a polypeptide of SEQ ID NO: 1.

The present disclosure also provides a compound (such as, an antibody or antigen binding fragment thereof) which specifically binds to an IL-3Rα chain, wherein the level of binding of the compound to a polypeptide comprising a sequence set forth in SEQ ID NO: 1 comprising one or more (or all) of the following combinations of amino acid substitutions:

(i) glutamic acid at position 51 is substituted with glutamine and serine at position 59 is substituted with alanine;
(ii) glutamic acid at position 51 is substituted with glutamine and proline at position 61 is substituted with threonine;
(iii) serine at position 59 is substituted with alanine and proline at position 61 is substituted with threonine; and/or
(iv) serine at position 59 is substituted with alanine and proline at position 89 is substituted with threonine,

is lower than the level of binding of the protein to a polypeptide of SEQ ID NO: 1.

The present disclosure provides a compound (such as an antibody or antigen binding fragment thereof) which specifically binds to an IL-3Rα chain, wherein the level of binding of the compound to a polypeptide comprising a sequence set forth in SEQ ID NO: 1 comprising one or more (or all) of the following amino acid substitutions:

(i) glutamic acid at position 51 is substituted with alanine or a conservative amino acid change; and/or
(ii) serine at position 59 is substituted with alanine or a conservative amino acid change;

is lower than the level of binding of the protein to a polypeptide of SEQ ID NO: 1.

The present disclosure also provides a compound (such as an antibody or antigen binding fragment thereof) which specifically binds to an IL-3Rα chain, wherein the level of binding of the compound to a polypeptide comprising a sequence set forth in SEQ ID NO: 1 comprising two or more (or all) of the following amino acid substitutions:

(i) glutamic acid at position 51 is substituted with alanine or a conservative amino acid change; and/or
(ii) serine at position 59 is substituted with alanine or a conservative amino acid change,

is lower than the level of binding of the protein to a polypeptide of SEQ ID NO: 1.

The present disclosure also provides a compound (such as an antibody or antigen binding fragment thereof) which specifically binds to an IL-3Rα chain, wherein the compound does not detectably bind to a polypeptide comprising a sequence set forth in SEQ ID NO: 1 comprising the following amino acid substitutions:

(i) glutamic acid at position 51 is substituted with alanine or a conservative amino acid change; and
(ii) serine at position 59 is substituted with alanine or a conservative amino acid change.

In one example, a compound of the present disclosure has one or more of the following characteristics:

(i) the compound binds to a polypeptide comprising a sequence set forth in SEQ ID NO: 1 in which the lysine at position 54 is substituted with alanine; and/or
(ii) the compound binds to a polypeptide comprising a sequence set forth in SEQ ID NO: 1 in which the proline at position 89 is substituted with alanine.

In one example, a compound that binds at a lower level to a protein comprising a substitution(s) binds at a level 20% or 30% or 40% or 50% or 60% or 70% or 80% or 90% or 95% lower than it binds to a protein comprising SEQ ID NO: 1 or a cell expressing same.

In one example, a compound that binds at a lower level to a protein comprising a substitution(s) does not detectably bind to the protein comprising the substitution(s). In one example, the level of binding is detected by Western blotting or by fluorescence activated cell sorting (FACS) analysis of cells expressing the protein comprising the substitution(s).

In one example, a compound of the disclosure detectably binds to a cell expressing SEQ ID NO: 1 and, optionally to a cell expressing SEQ ID NO: 1 and SEQ ID NO: 12 (IL-3Rβ chain), but does not detectably bind to a cell expressing SEQ ID NO: 1 comprising the substitution(s).

In one example, the compound makes contact with at least residues corresponding to amino acids 51 and 59 of SEQ ID NO: 1.

In one example, the compound makes contact with at least residues corresponding to amino acids 51, 58, 59 61, 84 and 89 of SEQ ID NO: 1.

In one example, the epitope bound by a compound of the disclosure is a conformational epitope.

In one example, a compound of the present disclosure competitively inhibits the binding of one or more of the following antibodies to a protein comprising a sequence set forth in SEQ ID NO: 1 or a cell expressing same:

(i) a V_{H} comprising a sequence set forth in SEQ ID NO: 2 and a light chain variable region (V_{L}) comprising a sequence set forth in SEQ ID NO: 3;
(ii) a V_{H} comprising complementarity determining regions (CDRs) of a sequence set forth in SEQ ID NO: 2 and a V_{L} comprising CDRs of a sequence set forth in SEQ ID NO: 3; and/or
(iii) a V_{H} comprising a sequence set forth in SEQ ID NO: 4 and a V_{L} comprising a sequence set forth in SEQ ID NO: 5.

In one example, a compound of the present disclosure neutralizes IL-3 signaling.

Compounds contemplated by the present disclosure can take any of a variety of forms including natural compounds, chemical small molecule compounds or biological compounds. Exemplary compounds include a nucleic acid (e.g., an aptamer), a polypeptide, a peptide, a small molecule, an antibody or an antigen binding fragment of an antibody.

In one example, the compound is a protein-based compound, e.g., a peptide, polypeptide or protein.

In one example, the compound is a protein comprising an antigen binding domain of an immunoglobulin, e.g., an IgNAR, a camelid antibody or a T cell receptor.

In another example, the compound is a protein comprising a non-antibody antigen binding domain, such as an adnectin, an affibody, an atrimer, an evasin, a designed ankyrin-repeat protein (DARPin) or an anticalin.

In one example, a compound of the present disclosure is an antibody or an antigen binding fragment thereof. In one example, an antibody of the present disclosure is a monoclonal antibody, a chimeric antibody, a humanized antibody or a human antibody.

In one example, an antibody or antigen binding fragment of the present disclosure is a human antibody or antigen binding fragment thereof.

Exemplary antigen binding fragments contemplated by the present disclosure include:

(i) a domain antibody (dAb);

### (ii) a Fv;

(iii) a scFv or stabilized form thereof (e.g., a disulfide stabilized scFv);
(iv) a dimeric scFv or stabilized form thereof;
(v) a diabody, triabody, tetrabody or higher order multimer;
(vi) Fab fragment;
(vii) a Fab′ fragment;
(viii) a F(ab′) fragment;
(ix) a F(ab′)₂ fragment;
(x) any one of (i)-(ix) fused to a Fc region of an antibody;
(xi) any one of (i)-(ix) fused to an antibody or antigen binding fragment thereof that binds to an immune effector cell.

In one example, a compound (e.g., an antibody or antigen binding fragment thereof) of the present disclosure depletes or at least partly eliminates cells to which it binds, e.g., leukemic cells and/or basophils and/or pDCs.

Exemplary compounds (e.g., antibodies) are capable of depleting or at least partly eliminating cells to which it binds without being conjugated to a toxic compound.

In one example, a compound (e.g., an antibody or antigen binding fragment thereof) of the present disclosure is capable of inducing an effector function, e.g., an effector function that results in killing a cell to which the antibody or antigen binding fragment thereof binds. Exemplary effector functions include ADCC, antibody-dependent cell-mediated phagocytosis (ADCP) and/or complement-dependent cytotoxicity (CDC).

In one example, the compound (e.g., the antibody or antigen binding fragment thereof) is capable of inducing ADCC.

In one example, a compound (e.g., an antibody or antigen binding fragment thereof) of the present disclosure comprises an antibody Fc region capable of inducing an effector function. For example, the effector function is Fc-mediated effector function. In one example, the Fc region is an IgG1 Fc region or an IgG3 Fc region or a hybrid IgG1/IgG2 Fc region.

In one example, a compound (e.g., an antibody or antigen binding fragment thereof) of the present disclosure is capable of inducing a similar (e.g., not significantly different or within about 10%) or the same level of effector function as a wild-type human IgG1 and/or human IgG3 Fc region.

In one example, the compound is capable of inducing an enhanced level of effector function.

In one example, the level of effector function induced by a compound comprising an Fc region is enhanced relative to that of the compound when it comprises a wild-type IgG1 Fc region

In one example, the level of effector function induced by an antibody or antigen binding fragment thereof of the disclosure is enhanced relative to that of the antibody or antigen binding fragment thereof when it comprises a wild-type IgG1 Fc region.

In one example, the compound comprises a Fc region that is afucosylated.

In one example, an antibody or antigen binding fragment thereof is afucosylated or comprises a Fc region that is afucosylated.

In another example, the compound (or antibody or antigen binding fragment thereof) comprises an Fc region having a lower level of fucosylation compared to an antibody or antigen binding fragment thereof or the Fc region when produced by a human or a CHO cell that has not been altered to reduce the level of fucosylation of proteins. In accordance with this example, a lower level of fucosylation will be understood to mean that in a composition comprising the compound (or antibody or antigen binding fragment thereof) the percentage of fucosylated compounds (or antibodies or fragments) (e.g., glycosyl groups attached to Asn297 of an antibody comprising fucose) is lower than produced by a human or a CHO cell that has not been altered to reduce the level of fucosylation of proteins.

In one example, the compound (e.g., antibody or antigen binding fragment thereof) comprises an Fc region comprising one or more amino acid sequence substitutions that enhance the effector function induced by the compound (e.g., antibody or antigen binding fragment). For example, the one or more amino acid sequence substitutions increase the affinity of the Fc region for a Fcγ receptor (FcγR) compared to a Fc region not comprising the substitutions. For example, the one or more amino acid substitutions enhance increase the affinity of the Fc region for a FcγR selected from the group consisting of FcγRI, FcγRIIa, FcγRIIc and FcγRIIIa compared to a Fc region not comprising the substitutions. In one example, the one or more amino acid sequence substitutions are:

(i) S239D, A330L and I332E according to the EU numbering system of Kabat; or
(ii) S239D and I332E according to the EU numbering system of Kabat.

In one example, an antibody or antigen binding fragment thereof of the present disclosure is a naked antibody or antigen binding fragment thereof.

In one example, an antibody of the present disclosure is a full length antibody.

In one example, a compound (e.g., an antibody or antigen binding fragment thereof) of the present disclosure binds to an IL-3Rα chain with an equilibrium dissociation constant (K_{D}) of 1×10⁻⁶ or less or 1×10⁻⁷ or less or 1×10⁻⁸M or less, such as 5×10⁻⁹M or less, for example, 3×10⁻⁹M or less, such as 2.5×10⁻⁹M or less.

In one example, a compound (e.g., an antibody or antigen binding fragment thereof) of the present disclosure binds to an IL-3Rα chain with a K_{D} of about 2.2×10⁻⁹M or less. In one example, the K_{D} is between about 1×10⁻⁹M and about 2.5×10⁻⁹M, for example is about 2.2×10⁻⁹M.

In one example, a compound (e.g., an antibody or antigen binding fragment thereof) of the present disclosure binds to an IL-3Rα chain with a K_{D} of about 9×10⁻¹⁰M or less, for example, about 8×10⁻¹⁰M or less. In one example, the K_{D} is between about 5×10⁻¹⁰M and about 9×10⁻¹⁰M, for example is about 7.8×10⁻¹⁰M.

In one example, the K_{D} is determined by Surface Plasmon Resonance. For example, soluble IL-3Rα (e.g., comprising the extracellular domain of IL-3Rα) is immobilized on a solid substrate and binding of the compound determined by Surface Plasmon Resonance.

The present disclosure also provides a composition comprising a compound (e.g., an antibody or antigen binding fragment thereof) according to the present disclosure and a pharmaceutically acceptable carrier.

The present disclosure also provides an isolated nucleic acid encoding a compound (e.g., a peptide or polypeptide compound or an antibody or antigen binding fragment thereof) of the present disclosure.

The present disclosure also provides an expression construct comprising an isolated nucleic acid of the disclosure operably linked to a promoter. In one example, the expression construct is an expression vector.

In one example, the expression construct of the disclosure comprises a nucleic acid encoding a polypeptide (e.g., comprising a V_{H}) operably linked to a promoter and a nucleic acid encoding another polypeptide (e.g., comprising a V_{L}) operably linked to a promoter.

In another example, the expression construct is a bicistronic expression construct, e.g., comprising the following operably linked components in 5′ to 3′ order:

(i) a promoter
(ii) a nucleic acid encoding a first polypeptide;
(iii) an internal ribosome entry site; and
(iv) a nucleic acid encoding a second polypeptide.

For example, the first polypeptide comprises a V_{H} and the second polypeptide comprises a V_{L}, or the first polypeptide comprises a V_{L} and the second polypeptide comprises a V_{H}.

The present disclosure also contemplates separate expression constructs one of which encodes a first polypeptide (e.g., comprising a V_{H}) and another of which encodes a second polypeptide (e.g., comprising a V_{L}). For example, the present disclosure also provides a composition comprising:

(i) a first expression construct comprising a nucleic acid encoding a polypeptide (e.g., comprising a V_{H}) operably linked to a promoter; and
(ii) a second expression construct comprising a nucleic acid encoding a polypeptide (e.g., comprising a V_{L}) operably linked to a promoter.

The disclosure also provides a host cell comprising an expression construct according to the present disclosure.

The present disclosure also provides an isolated cell expressing a compound (e.g., a peptide or polypeptide compound or an antibody or antigen binding fragment thereof of the disclosure or a recombinant cell genetically-modified to express the compound.

In one example, the cell comprises the expression construct of the disclosure or:

(i) a first genetic construct comprising a nucleic acid encoding a polypeptide (e.g., comprising a V_{H}) operably linked to a promoter; and
(ii) a second genetic construct comprising a nucleic acid encoding a polypeptide (e.g., comprising a V_{L}) operably linked to a promoter,
wherein the first and second polypeptides form an antibody or antigen binding fragment of the present disclosure.

The genetic construct can be integrated into the cell or remain episomal.

Examples of cells of the present disclosure include bacterial cells, yeast cells, insect cells or mammalian cells.

The present disclosure additionally provides a method for producing a compound (e.g., a peptide or polypeptide compound or an antibody or antigen binding fragment thereof) of the disclosure, the method comprising maintaining the genetic construct(s) of the disclosure under conditions sufficient for the compound to be produced.

In one example, the method for producing a compound of the disclosure comprises culturing the cell of the disclosure under conditions sufficient for the compound to be produced and, optionally, secreted.

In one example, the method for producing a compound of the disclosure additionally comprises isolating the compound thereof.

In one example, a method for producing a compound of the disclosure additionally comprises formulating the compound with a pharmaceutically acceptable carrier.

The present disclosure also provides a method of prophylactic or therapeutic treatment of a disease or condition in a mammal, the method comprising administering a compound (such as an antibody or antigen binding fragment thereof) of the disclosure to the mammal to thereby treat or prevent the disease or condition.

In one example, the mammal is a human.

In one example, the mammal is in need of treatment or prophylaxis.

In one example, the mammal in need suffers from the disease or condition.

In one example, the mammal in need is at risk of developing the disease or condition or a relapse thereof.

In one example, the subject has previously been shown to suffer from a disease or condition characterized by expression of IL-3Rα that is bound by an antibody that binds to one or more of the following:

(i) a peptide comprising a region bound by the compound (e.g., an antibody or antigen binding fragment thereof) of the disclosure, the region consisting of an amino acid sequence set forth between amino acids 51 to 89 of SEQ ID NO: 1;
(ii) an epitope within an IL-3Rα chain comprising the following:

(a) amino acid 51 of SEQ ID NO: 1;

(b) one or more (or all) of amino acids 58, 59 and/or 61 of SEQ ID NO: 1; and

(c) one or more (or all) of amino acids 84 and/or 89 of SEQ ID NO: 1; and/or

(iii) an epitope within an IL-3Rα chain comprising residues corresponding to amino acids 51, 58, 59 61, 84 and 89 of SEQ ID NO: 1.

The present disclosure also provides for use of a compound (e.g., an antibody or antigen binding fragment thereof) of the disclosure or a composition of the disclosure in medicine.

The present disclosure additionally or alternatively provides for use of a compound (e.g., an antibody or antigen binding fragment thereof) of the disclosure in the manufacture of a medicament for the treatment of a disease or condition in a mammal.

The present disclosure also provides a compound (e.g., an antibody or antigen binding fragment thereof) of the disclosure for use in the treatment of a disease or condition in a mammal.

In one example, the compound (e.g., antibody or antigen binding fragment or composition) is for the treatment of a disease or condition in a mammal previously shown to suffer from a disease or condition characterized by expression of IL-3Rα that is bound by an antibody that binds to one or more of the following:

(i) a peptide comprising a region bound by the compound (e.g., an antibody or antigen binding fragment thereof) of the disclosure, the region consisting of an amino acid sequence set forth between amino acids 51 to 89 of SEQ ID NO: 1;
(ii) an epitope within an IL-3Rα chain comprising residues positioned within each of the following regions:

(a) amino acid 51 of SEQ ID NO: 1;

(b) one or more (or all) of amino acids 58, 59 and/or 61 of SEQ ID NO: 1; and

(c) one or more (or all) of amino acids 84 and/or 89 of SEQ ID NO: 1; and/or

(iii) an epitope within an IL-3Rα chain comprising residues corresponding to amino acids 51, 58, 59 61, 84 and 89 of SEQ ID NO: 1.

In one example, the disease or condition is an IL-3Rα-mediated disease or condition.

In one example, the disease or condition is cancer or an autoimmune or inflammatory condition.

In one example, the disease or condition is myelodysplastic syndrome.

In one example, the disease or condition is cancer, such as a hematologic cancer, for example, leukemia, such as an acute leukemia (e.g., acute myelogenous leukemia) or a chronic leukemia (e.g., chronic myelomonocytic leukemia).

In one example, the disease or condition is an IL-3Rα-associated cancer, e.g., leukemia, i.e., the cancer (or leukemia) is characterized by cancer (or leukemia) cells expressing IL-3Rα.

In another example, the cancer is a malignant lymphoproliferative disorder such as lymphoma.

In one example, the disease or condition is an autoimmune condition or an inflammatory condition. For example, the condition is lupus, e.g., systemic lupus erythrematosus, Sjögren's syndrome or scleroderma (e.g., systemic scleroderma).

In one example, the method comprises administering an effective amount of the compound (e.g., the antibody or antigen binding fragment), such as a therapeutically effective amount of the compound.

The present disclosure also provides a peptide comprising a region bound by the compound of the disclosure, for example, the region consists of:

(i) an amino acid sequence set forth between amino acids 51-89 of SEQ ID NO: 1.
(ii) one or more amino acid sequences comprising residues positioned within each of the following regions:

(a) amino acid 51 of SEQ ID NO: 1;

(b) one or more (or all) of amino acids 58, 59 and/or 61 of SEQ ID NO: 1; and

(c) one or more (or all) of amino acids 84 and/or 89 of SEQ ID NO: 1; and/or

(iii) one or more amino acid sequences comprising residues corresponding to amino acids 51, 58, 59 61, 84 and 89 of SEQ ID NO: 1.

The present disclosure also provides a method of producing a compound (e.g., an antibody or antigen binding fragment thereof), the method comprising selecting a compound or a cell expressing same or particle displaying same that binds to IL-3Rα chain and one or more of the following:

(i) the peptide of the present disclosure;
(ii) an epitope within an IL-3Rα chain comprising residues positioned within each of the following regions:

(a) amino acid 51 of SEQ ID NO: 1;

(b) one or more (or all) of amino acids 58, 59 and/or 61 of SEQ ID NO: 1; and

(c) one or more (or all) of amino acids 84 and/or 89 of SEQ ID NO: 1; and/or

(iii) an epitope within an IL-3Rα chain comprising residues corresponding to amino acids 51, 58, 59 61, 84 and 89 of SEQ ID NO: 1.

In one example, the method additionally comprises selecting compound that neutralizes IL-3 signaling.

In one example, the compound is an antibody or antigen binding fragment thereof. In one example, the antibody or antigen binding fragment is chimeric, humanized or human.

In one example, the method additionally comprises reformatting the antigen binding fragment to thereby produce an antibody.

In one example, the method additionally comprises manufacturing the compound and, optionally, preparing a composition comprising the compound and a pharmaceutically acceptable carrier.

### BRIEF DESCRIPTION OF DRAWINGS

FIG. 1A is a diagrammatic representation showing proteins used to screen anti-IL-3Rα antibodies to identify regions bound. Proteins depicted are full length human IL-3Ra or proteins comprising hIL-3Ra domains (grey) with various GM-CSFRa domains (white).

FIG. 1B is a graphical representation showing the amino acid sequence of human IL-3Rα (SEQ ID NO: 1) and indicating the site of domains within the receptor. Vertical lines denote boundaries between the signal peptide sequence (boxed) and N-terminal domain (bold), and between the other domains indicated, i.e., two haemopoietin domains, transmembrane domain (underlined) and the cytoplasmic domain.

FIG. 2A is a graphical representation showing the level of binding of antibody 7G3 to IL-3Rα or alanine scanning mutants thereof as a percentage of control antibody (9F5, dark bars or 107D2, light bars). Percentage binding was determined as discussed in Example 1. The experiment was repeated 3 times and a representative histogram is shown. Arrows indicate residues that appear to significantly reduce 7G3 binding when mutated that do not appear to substantially affect binding of control antibodies.

FIG. 2B is a graphical representation showing the level of binding of antibody 7G3 to IL-3Rα or mutants thereof comprising conservative point mutations as a percentage of control antibody (9F5, dark bars or 107D2, light bars). Percentage binding was determined as discussed in Example 1. The experiment was repeated 3 times and a representative histogram is shown. Arrows indicate residues that appear to significantly reduce 7G3 binding when mutated that do not appear to substantially affect binding of control antibodies.

FIG. 2C contains copies of photographic representations showing results of Western blotting to detect binding of 7G3 or 9F5 (as indicated) to alanine scanning mutants of human IL-3Rα. Asterisks denote poorly expressed IL-3Rα mutants and triangles indicate mutants with differential 7G3/9F3 binding. (+) and (−) indicate positive (wild-type human IL-3Rα) and negative (wild-type human GM-CSFRα) controls, respectively.

FIG. 2D contains copies of photographic representations showing results of Western blotting to detect binding of 7G3 or 9F5 (as indicated) to conservative amino acid point mutants of human IL-3Rα. Asterisks denote poorly expressed IL-3Rα mutants and triangles indicate mutants with differential 7G3/9F3 binding. (+) and (−) indicate positive (wild-type human IL-3Rα) and negative (wild-type human GM-CSFRα) controls, respectively.

FIG. 3A contains copies of photographic representations showing results of Western blotting to detect binding of antibody CSL362, anti-HIS antibody or antibody S-20 (as indicated) to alanine scanning mutants of a fragment of human IL-3Rα comprising amino acids 20-59 of SEQ ID NO: 1. Asterisks denote poorly expressed IL-3Rα mutants and triangles indicate mutants with differential CSL362 binding. (+) and (−) indicate positive (wild-type human IL-3Rα) and negative (wild-type human GM-CSFRα) controls, respectively.

FIG. 3B contains copies of photographic representations showing results of Western blotting to detect binding of antibody CSL362, anti-HIS antibody or antibody S-20 (as indicated) to conservative amino acid point mutants of a fragment of human IL-3Rα comprising amino acids 60-100 of SEQ ID NO: 1. Asterisks denote poorly expressed IL-3Rα mutants and triangles indicate mutants with differential CSL362 binding. (+) and (−) indicate positive (wild-type human IL3-Rα) and negative (wild-type human GM-CSFRα) controls, respectively.

FIG. 4A contains a series of graphical representations showing the level of binding of antibodies 7G3, 9F5 or 107D2 to cells expressing wild type IL-3Rα or mutant forms thereof (as indicated). The experiment was repeated 3 times and representative histograms are shown.

FIG. 4B contains copies of photographic representations showing representative results of Western blotting to detect binding of antibodies 7G3 and 9F5 to wild type IL-3Rα or mutant forms thereof (as indicated).

FIG. 5 contains a series of graphical representations showing the level of binding of antibodies CSL362, 7G3, 9F5 or 107D2 to cells expressing wild type IL-3Rα or mutant forms thereof (as indicated). The experiment was repeated 3 times and representative histograms are shown.

FIG. 6 is a graphical representation showing a heatmap depicting levels of 7G3 binding to cell surface expressed mutants of IL-3Rα relative to levels of binding by non-neutralizing antibody 9F5. The level of 7G3 binding is expressed as a percentage of that observed with 9F5 and is represented as a heatmap. Results shown are representative of three independent experiments.

FIG. 7 is a graphical representation showing the amino acid sequence of human IL-3Rα (SEQ ID NO: 1) and indicating the regions that interact with antibody CSL362 as determined by X-Ray crystallography (bold text) and mutagenesis (italics and shading)..

### KEY TO SEQUENCE LISTING

SEQ ID NO: 1 is an amino acid sequence of human IL-3Rα.
SEQ ID NO: 2 is an amino acid sequence of the V_{H} of 7G3.
SEQ ID NO: 3 is an amino acid sequence of the V_{L} of 7G3.
SEQ ID NO: 4 is an amino acid sequence of the V_{H} of CSL362.
SEQ ID NO: 5 is an amino acid sequence of the V_{L} of CSL362.
SEQ ID NO: 6 is an amino acid sequence of HCDR1 of CSL362.
SEQ ID NO: 7 is an amino acid sequence of HCDR2 of CSL362.
SEQ ID NO: 8 is an amino acid sequence of HCDR3 of CSL362.
SEQ ID NO: 9 is an amino acid sequence of LCDR1 of CSL362.
SEQ ID NO: 10 is an amino acid sequence of LCDR2 of CSL362.
SEQ ID NO: 11 is an amino acid sequence of LCDR3 of CSL362.
SEQ ID NO: 12 is an amino acid sequence of human CD131.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, antibodies and fragments thereof herein may be further clarified by the discussion in Kabat Sequences of Proteins of Immunological Interest, National Institutes of Health, Bethesda, Md., 1987 and 1991.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65 of SEQ ID NO: 1” will be understood to mean that the region comprises a sequence of amino acids as numbered 56, 57, 58, 59, 60, 61, 62, 63, 64 and 65 in SEQ ID NO: 1.

### Selected Definitions

For the purposes of nomenclature only and not limitation, the amino acid sequence of an IL-3Rα chain is taught in Gene ID Accession Number 3563 and/or in SEQ ID NO: 1. In one example, the IL-3Rα is human IL-3Rα. The IL-3 receptor comprises 280 aminoacids arranged in a cytokine receptor module (CRM) and an N-terminal domain (NTD) with an Ig like-fold.

The term “immunoglobulin” will be understood to include any antigen binding protein comprising an immunoglobulin domain. Exemplary immunoglobulins are antibodies. Additional proteins encompassed by the term “immunoglobulin” include domain antibodies, camelid antibodies and antibodies from cartilaginous fish (i.e., immunoglobulin new antigen receptors (IgNARs)). Generally, camelid antibodies and IgNARs comprise a V_{H}, however lack a V_{L} and are often referred to as heavy chain immunoglobulins. Other “immunoglobulins” include T cell receptors.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region or constant fragment or fragment crystallizable (Fc). A V_{H} and a V_{L} interact to form a Fv comprising an antigen binding region that specifically binds to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof. In some cases, the intact antibody may be capable of inducing one or more effector functions.

The term “naked antibody” refers to an antibody that is not conjugated to another compound, e.g., a toxic compound or radiolabel.

An “antigen binding fragment” of an antibody comprises one or more variable regions of an intact antibody. Examples of antibody fragments include Fab, Fab′, F(ab′)₂ and Fv fragments; diabodies; linear antibodies; single-chain antibody molecules and multispecific antibodies formed from antibody fragments.

In the context of the present disclosure, “effector functions” refer to those biological activities mediated by cells or proteins that bind to the Fc region (a native sequence Fc region or amino acid sequence variant Fc region) of an antibody that result in killing of a cell. Examples of effector functions induced by antibodies or antigen binding fragments thereof include: complement dependent cytotoxicity; antibody-dependent-cell-mediated cytotoxicity (ADCC); antibody-dependent-cell-phagocytosis (ADCP); and B-cell activation.

“Antibody-dependent-cell-mediated cytotoxicity” or “ADCC” refers to a form of cytotoxicity in which secreted Ig bound onto Fc receptors (“FcRs”) present on certain cytotoxic cells (e.g., natural killer (“NK”) cells, neutrophils and macrophages) enable these cytotoxic effector cells to bind specifically to an antigen-bearing target-cell and subsequently kill the target-cell with cytotoxins. To assess ADCC activity of a molecule of interest, an in vitro ADCC assay may be performed. Useful effector cells for such assays include peripheral blood mononuclear cells (“PBMC”) and NK cells.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”. According to the numbering system of Kabat, V_{H} FRs and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4).

“Framework regions” (hereinafter FR) are those variable domain residues other than the CDR residues.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy (C_{H})1, a linker, a C_{H}2 and a C_{H}3. In a heavy chain, a constant region comprises a Fc. In a light chain, a constant region generally comprise one constant domain (a C_{L}1).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Exemplary heavy chain constant regions are gamma 1 (IgG1), gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of γ, α or δ heavy chain comprises two constant domains.

The term “EU numbering system of Kabat” will be understood to mean the numbering of an antibody heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU antibody.

Reference herein to “monoclonal antibody 7G3” or to “7G3” is a reference to the monoclonal antibody produced by the hybridoma designated 7G3 as deposited with the ATCC under accession number HB-12009 and described in U.S. Pat. No. 6,177,078. Monoclonal antibody 7G3 is also commercially available, e.g., from BD Biosciences (NJ, USA).

As used herein, the term “binds” in reference to the interaction of a compound with an antigen means that the interaction is dependent upon the presence of a particular structure (e.g., an antigenic determinant or epitope) on the antigen. For example, a compound, such as an antibody, recognizes and binds to a specific protein structure rather than to proteins generally. If a compound binds to epitope “A”, the presence of a molecule containing epitope “A” (or free, unlabeled “A”), in a reaction containing labeled “A” and the compound, will reduce the amount of labeled “A” bound to the compound.

As used herein, the term “specifically binds” shall be taken to mean that the binding interaction between an antibody or antigen binding fragment thereof and IL-3Rα chain is dependent on the presence of the antigenic determinant or epitope of an IL-3Rα chain bound by the antibody or antigen binding fragment thereof. Accordingly, the antibody or antigen binding fragment thereof preferentially binds or recognizes an IL-3Rα chain antigenic determinant or epitope even when present in a mixture of other molecules or organisms. In one example, the antibody or antigen binding fragment thereof reacts or associates more frequently, more rapidly, with greater duration and/or with greater affinity with IL-3Rα or cell expressing same than it does with alternative antigens or cells. It is also understood by reading this definition that, for example, an antibody or antigen binding fragment thereof specifically binds to IL-3Rα may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. “. Methods for determining specific binding will be apparent to the skilled person. For example, a compound of the disclosure is contacted with IL-3Rα or a cell expressing same or a mutant form thereof or an alternative antigen. The binding of the compound to the IL-3Rα or mutant form or alternative antigen is then determined and a compound that binds as set out above to the IL-3Rα rather than the mutant or alternative antigen is considered to specifically bind to IL-3Rα. In one example, “specific binding” to with IL-3Rα or cell expressing same, means that the antibody or antigen binding fragment binds to the with IL-3Rα or cell expressing same with an equilibrium constant (K_{D}) of 1 μM or less, such as 100 nM or less, such as 50 nM or less, for example 20 nM or less, such as, 1 nM or less, e.g., 0.8 nM or less. 1×10⁻⁸M or less, such as 5×10⁻⁹M or less, for example, 3×10⁻⁹M or less, such as 2.5×10⁻⁹M or less. In one example, the K_{D} is about 2.2×10⁻⁹M or less, for example, 1×10⁻⁹M or less, for example, 9×10⁻¹⁰M or less, for example, about 8×10⁻¹⁰M or less. In one example, the K_{D} is determined by Surface Plasmon Resonance. For example, soluble IL-3Rα (e.g., comprising the extracellular domain of IL-3Rα) is immobilized on a solid substrate and binding of the compound determined by Surface Plasmon Resonance.

As used herein, the term “does not detectably bind” shall be understood to mean that an antibody or antigen binding fragment thereof binds to a candidate antigen (e.g., IL-3Rα) at a level less than 10%, or 8% or 6% or 5% above background. The background can be the level of binding signal detected in the absence of the antibody or antigen binding fragment and/or in the presence of a negative control protein (e.g., an isotype control antibody) and/or the level of binding detected in the presence of a negative control antigen. The level of binding is detected using Western Blotting and/or FACS analysis of cells expressing IL-3Rα or lacking expression of IL-3Rα.

As used herein, phrases referring to “reduced binding” or “binding being at a lower level” in relation to an antigen will be understood to mean that an antibody binds to an antigen (e.g., an alanine point mutant of SEQ ID NO:1) with an affinity at least about 5 fold or 10 fold or 20 fold or 40 fold or 60 fold less than a control epitope or antigen (e.g. SEQ ID NO:1). In one example, the level of binding is determined by Surface Plasmon Resonance, e.g., as described herein. In another example, the level of binding is determined by FACS. For example, a cell expressing the antigen is contacted with a labeled compound (e.g., a fluorescently labelled compound) and the amount of fluorescence on the surface of the cell determined by FACS. The level of fluorescence is considered to be related to the amount of binding of the compound. By determining the level of fluorescence in the presence and absence of the compound and the amount of fluorescence on a cell expressing a protein having the sequence set forth in SEQ ID NO: 1, a compound that binds at a reduced level can be determined. In a further example, the level of binding is determined by Western blotting. For example, a recombinant mutant of IL-3Rα as described herein and recombinant IL-3Rα are resolved by SDS-PAGE and then contacted with labelled compound (or contacted with compound, which is then contacted with a labelled detection reagent that binds to the compound). The level of label bound is then determined (and, optionally, normalized for total amount of protein resolved) and the amount of compound bound to the IL-3Rα and mutant form thereof determined.

As used herein, the term “contact” will be understood to mean that a residue or chemical group of a compound of the disclosure interacts with a residue of IL-3Rα, e.g., by a hydrogen bond, an ionic bond, a Van der Waals force, or a hydrophobic interaction.

As used herein, the term “epitope” (syn. “antigenic determinant”) shall be understood to mean a region of IL-3Rα to which an antibody or antigen binding fragment thereof binds. This term is not necessarily limited to the specific residues or structure to which the antibody or antigen binding fragment makes contact. In some examples, the epitope comprises a series of discontinuous amino acids that are positioned close to one another when IL-3Rα is folded, i.e., a “conformational epitope”.

The term “competitively inhibits” shall be understood to mean that an antibody or antigen binding fragment thereof reduces or prevents binding of a recited antibody to IL-3Rα. It will be apparent from the foregoing that the antibody or antigen binding fragment thereof need not completely inhibit binding of the recited antibody, rather it need only reduce binding by a statistically significant amount, for example, by at least about 10% or 20% or 30% or 40% or 50% or 60% or 70% or 80% or 90% or 95%. Methods for determining competitive inhibition of binding are known in the art and/or described herein. For example, the antibody is exposed to IL-3Rα either in the presence or absence of a test antibody or antigen binding fragment thereof. If less of the antibody binds in the presence of the test antibody or antigen binding fragment than in the absence of the test antibody or antigen binding fragment, the antibody or antigen binding fragment is considered to competitively inhibit binding of the antibody.

By “overlapping” in the context of two epitopes shall be taken to mean that two epitopes share a sufficient number of amino acid residues to permit an antibody or antigen binding fragment that binds to one epitope to competitively inhibit the binding of an antibody or antigen binding fragment that binds to the other epitope. For example, the two epitopes share at least 1 or 2 or 3 or 4 or 5 or 6 or more amino acids.

By “individually” is meant that the disclosure encompasses a compound that binds to the recited antigens or groups of antigens separately, and that, notwithstanding that individual antigens or groups of antigens may not be separately listed herein the accompanying claims may define such antigens or groups of antigens separately and divisibly from each other.

By “collectively” is meant that the disclosure encompasses any number or combination of the recited antigens or groups of antigens, and that, notwithstanding that such numbers or combinations of antigens or groups of antigens may not be specifically listed herein the accompanying claims may define such combinations or sub-combinations separately and divisibly from any other combination of antigens or groups of antigens.

As used herein, the term “neutralize” shall be taken to mean that an antibody or antigen binding fragment thereof is capable of reducing or preventing IL-3-mediated signaling (syn. IL-3 signaling) in a cell and/or reducing or preventing IL-3 binding to IL-3Rα chain and/or a heterodimer of IL-3Rα chain and IL-3Rβ chain (also known as colony stimulating factor 2 receptor). Methods for determining whether or not a compound neutralizes IL-3 signaling will be apparent to the skilled artisan. For example, recombinant cells (e.g., 293F cells) expressing IL-3Rα and CD131 are contacted with the compound and subsequently or simultaneously (or previously) contacted with IL-3 (e.g., at a concentration 30 ng/ml) for about 20 min. STATS activity is then measured, e.g., by flow cytometry after intracellular staining with a phosphoSTAT5 antibody (pSTAT5). Reduced levels of STATS activity indicate that the compound neutralizes IL-3 signaling. In another example, Ba/F3 cells expressing IL-3Rα and CD131 are contacted with the compound subsequently or simultaneously (or previously) contacted with increasing concentrations of IL-3 for, e.g., about 72 hours and cell proliferation determined, e.g., by tritiated-thymidine incorporation. Reduced proliferation in the presence of the compound compared to the absence of the compound indicates that the compound neutralizes IL-3 signaling.

A “conservative amino acid substitution” is one in which the amino acid residue is replaced with an amino acid residue having a similar side chain and/or hydropathicity and/or hydrophilicity. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

As used herein, the term “IL-3Rα-mediated condition” will be understood to mean a condition associated with or caused by excessive IL-3Rα expression and/or an excessive number of IL-3Rα expressing cells in a mammal, such as cancer cells (e.g., leukemic cells) and/or immune cells (e.g., plasmacytoid dendritic cells).

As used herein, the term “myelodysplastic syndrome” or “MDS” will be understood to refer to a diverse collection of hematological medical conditions that involve ineffective production (or dysplasia) of the myeloid class of blood cells. Subjects with MDS often develop severe anemia and can require frequent blood transfusions. In many cases, as MDS progresses the subject develops cytopenias (low blood counts) due to progressive bone marrow failure. In about one third of patients with MDS, the disease transforms into acute myelogenous leukemia (AML). The MDS can be diagnosed or classified to various systems, including the French-American-British Classification System (Bennett et al., Br. J. Haematol. 33: 451-458, 1976), The International Prognostic Scoring System (Greenberg et al., Blood 89: 2079-88, 1997) or a system published by the World Health Organization.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease are mitigated or eliminated.

As used herein, the term “prevention” includes providing prophylaxis with respect to occurrence or recurrence of a disease in an individual. An individual may be predisposed to or at risk of developing the disease or disease relapse but has not yet been diagnosed with the disease or the relapse.

As used herein, a mammal “at risk” of developing a disease or condition or relapse thereof or relapsing may or may not have detectable disease or symptoms of disease, and may or may not have displayed detectable disease or symptoms of disease prior to the treatment according to the present disclosure. “At risk” denotes that a mammal has one or more risk factors, which are measurable parameters that correlate with development of the disease or condition, as known in the art and/or described herein.

An “effective amount” refers to at least an amount effective, at dosages and for periods of time necessary, to achieve the desired therapeutic or prophylactic result. An effective amount can be provided in one or more administrations. In some examples of the present disclosure, the term “effective amount” is meant an amount necessary to effect treatment of a disease or condition as hereinbefore described. The effective amount may vary according to the disease or condition to be treated and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. The effective amount can be administered in a single dose or in a dose repeated once or several times over a treatment period.

A “therapeutically effective amount” is at least the minimum concentration required to effect a measurable improvement of a particular disorder (e.g., SLE). A therapeutically effective amount herein may vary according to factors such as the disease state, age, sex, and weight of the patient, and the ability of the antibody or antigen binding fragment thereof to elicit a desired response in the individual. A therapeutically effective amount is also one in which any toxic or detrimental effects of the antibody or antigen binding fragment thereof are outweighed by the therapeutically beneficial effects.

A “prophylactically effective amount” refers to an amount effective, at the dosages and for periods of time necessary, to achieve the desired prophylactic result. Typically but not necessarily, since a prophylactic dose is used in mammals prior to or at an earlier stage of disease, a prophylactically effective amount may be less than a therapeutically effective amount.

The “mammal” treated according to the present disclosure may be a mammal, such as a non-human primate or a human. In one example, the mammal is a human.

### Compounds

As discussed herein, compounds of the present disclosure can take various forms, e.g., protein-based compounds or chemical compounds. Typically, the compounds are antibodies or antigen binding fragments thereof. Exemplary compounds are discussed herein.

### Antibodies

### Immunization-Based Methods

Methods for generating antibodies are known in the art and/or described in Harlow and Lane (editors) Antibodies: A Laboratory Manual, Cold Spring Harbor Laboratory, (1988). Generally, in such methods an IL-3Rα protein or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (Mabs).

Monoclonal antibodies are one exemplary form of antibody contemplated by the present disclosure. The term “monoclonal antibody” or “MAb” refers to a homogeneous antibody population capable of binding to the same antigen(s), for example, to the same epitope within the antigen. This term is not intended to be limited as regards to the source of the antibody or the manner in which it is made.

For the production of Mabs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human immunoglobulin proteins and, for example, do not express murine immunoglobulin proteins, can also be used to generate an antibody of the present disclosure (e.g., as described in WO2002/066630).

Following immunization, somatic cells with the potential for producing antibodies, specifically B lymphocytes (B cells), are selected for use in the MAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemistry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

### Library-Based Methods

The present disclosure also encompasses screening of libraries of antibodies or antigen binding fragments thereof (e.g., comprising variable regions thereof).

Examples of libraries contemplated by this disclosure include naïve libraries (from unchallenged subjects), immunized libraries (from subjects immunized with an antigen) or synthetic libraries. Nucleic acid encoding antibodies or regions thereof (e.g., variable regions) are cloned by conventional techniques (e.g., as disclosed in Sambrook and Russell, eds, Molecular Cloning: A Laboratory Manual, 3rd Ed, vols. 1-3, Cold Spring Harbor Laboratory Press, 2001) and used to encode and display proteins using a method known in the art. Other techniques for producing libraries of proteins are described in, for example in U.S. Pat. No. 6,300,064 (e.g., a HuCAL library of Morphosys AG); U.S. Pat. No. 5,885,793; U.S. Pat. No. 6,204,023; U.S. Pat. No. 6,291,158; or U.S. Pat. No. 6,248,516.

The antigen binding fragments according to the disclosure may be soluble secreted proteins or may be presented as a fusion protein on the surface of a cell, or particle (e.g., a phage or other virus, a ribosome or a spore). Various display library formats are known in the art. For example, the library is an in vitro display library (e.g., a ribosome display library, a covalent display library or a mRNA display library, e.g., as described in U.S. Pat. No. 7,270,969). In yet another example, the display library is a phage display library wherein proteins comprising antigen binding fragments of antibodies are expressed on phage, e.g., as described in U.S. Pat. No. 6,300,064; U.S. Pat. No. 5,885,793; U.S. Pat. No. 6,204,023; U.S. Pat. No. 6,291,158; or U.S. Pat. No. 6,248,516. Other phage display methods are known in the art and are contemplated by the present disclosure. Similarly, methods of cell display are contemplated by the disclosure, e.g., bacterial display libraries, e.g., as described in U.S. Pat. No. 5,516,637; yeast display libraries, e.g., as described in U.S. Pat. No. 6,423,538 or a mammalian display library.

Methods for screening display libraries are known in the art. In one example, a display library of the present disclosure is screened using affinity purification, e.g., as described in Scopes (In: Protein purification: principles and practice, Third Edition, Springer Verlag, 1994). Methods of affinity purification typically involve contacting proteins comprising antigen binding fragments displayed by the library with a target antigen (e.g., IL-3Rα) and, following washing, eluting those domains that remain bound to the antigen.

Any variable regions or scFvs identified by screening are readily modified into a complete antibody, if desired. Exemplary methods for modifying or reformatting variable regions or scFvs into a complete antibody are described, for example, in Jones et al., J Immunol Methods. 354:85-90, 2010; or Jostock et al., J Immunol Methods, 289: 65-80, 2004; or WO2012/040793. Alternatively, or additionally, standard cloning methods are used, e.g., as described in Ausubel et al (In: Current Protocols in Molecular Biology. Wiley Interscience, ISBN 047 150338, 1987), and/or (Sambrook et al (In: Molecular Cloning: Molecular Cloning: A Laboratory Manual, Cold Spring Harbor Laboratories, New York, Third Edition 2001).

### Deimmunized, Chimeric, Humanized, Synhumanized, Primatized and Human Antibodies or Antigen Binding Fragments

The antibodies or antigen binding fragments of the present disclosure may be may be humanized.

The term “humanized antibody” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized antibodies also include antibodies in which one or more residues of the human protein are modified by one or more amino acid substitutions and/or one or more FR residues of the human antibody are replaced by corresponding non-human residues. Humanized antibodies may also comprise residues which are found in neither the human antibody or in the non-human antibody. Any additional regions of the antibody (e.g., Fc region) are generally human. Humanization can be performed using a method known in the art, e.g., U.S. Pat. No. 5,225,539, U.S. Pat. No. 6,054,297, U.S. Pat. No. 7,566,771 or U.S. Pat. No. 5,585,089. The term “humanized antibody” also encompasses a super-humanized antibody, e.g., as described in U.S. Pat. No. 7,732,578. A similar meaning will be taken to apply to the term “humanized antigen binding fragment”.

The antibodies or antigen binding fragments thereof of the present disclosure may be human antibodies or antigen binding fragments thereof. The term “human antibody” as used herein refers to antibodies having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human antibody will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516. A similar meaning will be taken to apply to the term “human antigen binding fragment”.

The antibodies or antigen binding fragments thereof of the present disclosure may be synhumanized antibodies or antigen binding fragments thereof. The term “synhumanized antibody” refers to an antibody prepared by a method described in WO2007/019620. A synhumanized antibody includes a variable region of an antibody, wherein the variable region comprises FRs from a New World primate antibody variable region and CDRs from a non-New World primate antibody variable region.

The antibody or antigen binding fragment thereof of the present disclosure may be primatized. A “primatized antibody” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example an antibody or antigen binding fragment thereof of the disclosure is a chimeric antibody or fragment. The term “chimeric antibody” or “chimeric antigen binding fragment” refers to an antibody or fragment in which one or more of the variable domains is from a particular species (e.g., murine, such as mouse or rat) or belonging to a particular antibody class or subclass, while the remainder of the antibody or fragment is from another species (such as, for example, human or non-human primate) or belonging to another antibody class or subclass. In one example, a chimeric antibody comprising a V_{H} and/or a V_{L} from a non-human antibody (e.g., a murine antibody) and the remaining regions of the antibody are from a human antibody. The production of such chimeric antibodies and antigen binding fragments thereof is known in the art, and may be achieved by standard means (as described, e.g., in U.S. Pat. No. 6,331,415; U.S. Pat. No. 5,807,715; U.S. Pat. No. 4,816,567 and U.S. Pat. No. 4,816,397).

The present disclosure also contemplates a deimmunized antibody or antigen binding fragment thereof, e.g., as described in WO2000/34317 and WO2004/108158. De-immunized antibodies and fragments have one or more epitopes, e.g., B cell epitopes or T cell epitopes removed (i.e., mutated) to thereby reduce the likelihood that a subject will raise an immune response against the antibody or protein. For example, an antibody of the disclosure is analyzed to identify one or more B or T cell epitopes and one or more amino acid residues within the epitope is mutated to thereby reduce the immunogenicity of the antibody.

### Antibody Fragments

### Single-Domain Antibodies

In some examples, an antigen binding fragment of an antibody of the disclosure is or comprises a single-domain antibody (which is used interchangeably with the term “domain antibody” or “dAb”). A single-domain antibody is a single polypeptide chain comprising all or a portion of the heavy chain variable domain of an antibody.

### Diabodies, Triabodies, Tetrabodies

In some examples, an antigen binding fragment of the disclosure is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

A diabody, triabody, tetrabody, etc capable of inducing effector activity can be produced using an antigen binding fragment capable of binding to IL-3Rα and an antigen binding fragment capable of binding to a cell surface molecule on an immune cell, e.g., a T cell (e.g., CD3).

Single Chain Fv (scFv) Fragments

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present disclosure encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

The present disclosure also contemplates a dimeric scFv capable of inducing effector activity. For example, one scFv binds to IL-3Rα and comprises CDRs and/or variable regions described herein and another scFv binds to a cell surface molecule on an immune cell, e.g., a T cell (e.g., CD3 or CD19). In one example, the dimeric protein is a combination of a dAb and a scFv. Examples of bispecific antibody fragments capable of inducing effector function are described, for example, in U.S. Pat. No. 7,235,641.

### Other Antibodies and Antibody Fragments

The present disclosure also contemplates other antibodies and antibody fragments, such as:

(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### Exemplary Antibodies or Antigen Binding Fragments

In one example, the antibody or antigen binding fragment thereof is not:

(i) an antibody or antigen binding fragment thereof comprising a V_{H} comprising a sequence set forth in SEQ ID NO: 2 and a V_{L} comprising a sequence set forth in SEQ ID NO: 3; or
(ii) an antibody or antigen binding fragment thereof comprising a V_{H} comprising CDRs 1, 2 and 3 of a sequence set forth in SEQ ID NO: 2 and a V_{L} comprising CDRs 2 and 3 of a sequence set forth in SEQ ID NO: 3.

A mouse monoclonal antibody comprising a V_{H} comprising a sequence set forth in SEQ ID NO: 2 and a V_{L} comprising a sequence set forth in SEQ ID NO: 3 is also known as antibody 7G3 and was disclosed as binding to an epitope within residues 19-49 of IL-3Rα in U.S. Pat. No. 6,177,078 and Sun et al., Blood, 87: 83-92, 1996. Thus, these document teach that an antibody that neutralizes IL-3 signaling binds within a different region of IL-3Rα than the compounds of the present disclosure.

In one example, the antibody or antigen binding fragment thereof is not:

(i) an antibody or antigen binding fragment thereof comprising a V_{H} comprising a sequence set forth in SEQ ID NO: 2 and a V_{L} comprising a sequence set forth in SEQ ID NO: 3; or
(ii) an antibody or antigen binding fragment thereof comprising a V_{H} comprising complementarity determining regions (CDRs) 1, 2 and 3 of a sequence set forth in SEQ ID NO: 2 and a V_{L} comprising CDRs 2 and 3 of a sequence set forth in SEQ ID NO: 3;
(iii) an antibody or antigen binding fragment thereof, which is a humanized version of an antibody or antigen binding fragment thereof comprising a V_{H} comprising a sequence set forth in SEQ ID NO: 2 and a V_{L} comprising a sequence set forth in SEQ ID NO: 3 and having a K_{D} for IL-3Rα chain of 1.06 nM and an IC₅₀ of 19 nM as determined in an assay measuring IL-3-dependent TF-1 cell proliferation or having a K_{D} for IL-3Rα and an IC₅₀ of 6 nM as determined in an assay measuring IL-3-dependent TF-1 cell proliferation;
(iv) an antibody or antigen binding fragment thereof comprising a V_{H} comprising a sequence set forth in SEQ ID NO: 4;
(v) an antibody or antigen binding fragment thereof comprising a V_{L} comprising a sequence set forth in SEQ ID NO: 5;
(vi) an antibody or antigen binding fragment thereof comprising a V_{H} comprising CDRs1, 2 and 3 as set forth in SEQ ID Nos: 6, 7 and 8, respectively;
(vii) an antibody or antigen binding fragment thereof comprising a V_{L} comprising CDRs 1, 2 and 3 as set forth in SEQ ID Nos: 9, 10 and 11, respectively; and/or
(viii) an antibody or antigen binding fragment thereof comprising a V_{H} comprising CDRs 1, 2 and 3 as set forth in SEQ ID Nos: 6, 7 and 8, respectively and a V_{L} comprising CDRs 1, 2 and 3 as set forth in SEQ ID Nos: 9, 10 and 11, respectively.

In one example, the antibody or antigen binding fragment thereof does not comprise a V_{H} comprising CDRs 1, 2 and 3 as set forth in SEQ ID Nos: 6, 7 and 8, respectively and a V_{L} comprising CDRs 2 and 3 as set forth in SEQ ID Nos: 10 and 11, respectively.

In one example, the antibody or antigen binding fragment thereof does not comprise a V_{H} comprising one or more of CDRs 1, 2 and 3 as set forth in SEQ ID Nos: 6, 7 and 8, respectively and/or a V_{L} comprising one or more of CDRs 1, 2 and 3 as set forth in SEQ ID Nos: 9, 10 and 11, respectively.

In one example, the antibody or antigen binding fragment of the present disclosure does not comprise one or more of the CDRs of a monoclonal antibody comprising a V_{H} comprising a sequence set forth in SEQ ID NO: 2 or 4 and a V_{L} comprising a sequence set forth in SEQ ID NO: 3 or 5.

In one example, the antibody is not:

(i) a mouse monoclonal antibody comprising a V_{H} comprising a sequence set forth in SEQ ID NO: 2 and a V_{L} comprising a sequence set forth in SEQ ID NO: 3;
(ii) a chimeric antibody comprising a V_{H} comprising a sequence set forth in SEQ ID NO: 2 and a V_{L} comprising a sequence set forth in SEQ ID NO: 3; or
(iii) a humanized form of a mouse monoclonal antibody comprising a V_{H} comprising a sequence set forth in SEQ ID NO: 2 and a V_{L} comprising a sequence set forth in SEQ ID NO: 3.

In one example, the antibody is not monoclonal antibody 7G3 as described in U.S. Pat. No. 6,177,078 or CSL360, CSL360_{S239D/I332E}, CSL360_{S239D/A330L/1332E}, hCSL360, hCSL360_{S239D/I332E}, hCSL360_{S239D/A330L/1332E}, or 168-26 as described in WO2009/070844 or ch7G3, hz7G3, hz7G3V1, hz7G3V2 or hz7G3V3 as described in WO2011/100786 or CSL362, CSL362B, CSL362X1 or CSL362X2 as described in WO2012/021934 or the antigen binding fragment is not an antigen binding fragment of any one of the foregoing antibodies.

In one example, the antibody or antigen binding fragment thereof is not any one or more of the following:

(i) a monoclonal antibody or antibody fragment produced by the 7G3 hybridoma cell line as described in U.S. Pat. No. 6,177,078;
(ii) a monoclonal antibody or antibody fragment or peptide or peptide fragment or compound directed at amino acids 19-49 of IL-3Rα as described in U.S. Pat. No. 6,177,078;
(iii) an antibody, antibody fragment or compound as described in U.S. Pat. No. 6,177,078;
(iv) an antibody described in Sun et al., Blood, 87: 83-92, 1996;
(v) an antigen binding molecule as described in WO2009/070844;
(vi) an antigen binding molecule, immunoglobulin, antibody, antigen-binding and/or variable-domain-comprising fragment of an immunoglobulin or molecule as described in WO2009/070844;
(vii) an immunoglobulin as described in WO2011/100786;
(viii) an antibody or antigen binding fragment of an antibody as described in WO2011/100786;
(ix) an immunoglobulin as described in WO2012/021934; and/or
(viii) an antibody or antigen binding fragment of an antibody as described in WO2012/021934.

In one example, the antibody or antigen binding fragment is not 7G3 or a chimeric, humanized or affinity matured humanized form thereof.

In one example, a compound (or antibody or antigen binding fragment) of the disclosure binds to an epitope comprising the following regions:

(i) a region comprising amino acids 48 to 51 of SEQ ID NO: 1;
(ii) a region comprising amino acids 56 to 65 of SEQ ID NO: 1; and
(iii) a region comprising amino acids 84 to 91 of SEQ ID NO: 1.

### Immunoglobulins and Immunoglobulin Fragments

An example of a compound of the present disclosure is a protein comprising a variable region of an immunoglobulin, such as a T cell receptor or a heavy chain immunoglobulin (e.g., an IgNAR, a camelid antibody).

### Heavy Chain Immunoglobulins

Heavy chain immunoglobulins differ structurally from many other forms of immunoglobulin (e.g., antibodies), in so far as they comprise a heavy chain, but do not comprise a light chain. Accordingly, these immunoglobulins are also referred to as “heavy chain only antibodies”. Heavy chain immunoglobulins are found in, for example, camelids and cartilaginous fish (also called IgNAR).

The variable regions present in naturally occurring heavy chain immunoglobulins are generally referred to as “Vim domains” in camelid Ig and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

Heavy chain immunoglobulins do not require the presence of light chains to bind with high affinity and with high specificity to a relevant antigen. This means that single domain binding fragments can be derived from heavy chain immunoglobulins, which are easy to express and are generally stable and soluble.

A general description of heavy chain immunoglobulins from camelids and the variable regions thereof and methods for their production and/or isolation and/or use is found inter alia in the following references WO94/04678, WO97/49805 and WO 97/49805.

A general description of heavy chain immunoglobulins from cartilaginous fish and the variable regions thereof and methods for their production and/or isolation and/or use is found inter alia in WO2005/118629.

### V-Like Proteins

An example of a compound of the disclosure is a T-cell receptor. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999/045110.

### Adnectins

In one example, a compound of the disclosure is an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005/056764.

### Anticalins

In a further example, a compound of the disclosure is an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

### Affibodies

In a further example, a compound of the disclosure is an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

### Avimers

In a further example, a compound of the disclosure is an Avimer. Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see WO2002088171.

### DARPins

In a further example, a compound of the disclosure is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### Other Non Antibody Polypeptides

Other non-antibody proteins comprising binding domains include those based on human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins).

### Peptides

In one example, a binding molecule is a peptide, e.g., isolated from a random peptide library. To identify a suitable peptide, a random peptide library is generated and screened as described in U.S. Pat. No. 5,733,731, U.S. Pat. No. 5,591,646 and U.S. Pat. No. 5,834,318. Generally, such libraries are generated from short random oligonucleotides that are expressed either in vitro or in vivo and displayed in such a way to facilitate screening of the library to identify a peptide that. is capable of specifically binding to an antigen described herein. Methods of display include, phage display, retroviral display, bacterial surface display, bacterial flagellar display, bacterial spore display, yeast surface display, mammalian surface display, and methods of in vitro display including, mRNA display, ribosome display and covalent display.

A peptide that is capable of binding an antigen described herein is identified by any of a number of methods known in the art, such as, for example, standard affinity purification methods as described, for example in Scopes, 1994) purification using FACS analysis as described in U.S. Pat. No. 645,563.

### Small Molecules

In another example, a binding molecule is a small molecule. Such a small molecule may be isolated from a library. Chemical small molecule libraries are available commercially or alternatively may be generated using methods known in the art, such as, for example, those described in U.S. Pat. No. 5,463,564.

Techniques for synthesizing small organic compounds will vary considerably depending upon the compound, however such methods will be known to those skilled in the art.

In one example, informatics is used to select suitable chemical building blocks from known compounds, for producing a combinatorial library. For example, QSAR (Quantitative Structure Activity Relationship) modeling approach uses linear regressions or regression trees of compound structures to determine suitability. The software of the Chemical Computing Group, Inc. (Montreal, Canada) uses high-throughput screening experimental data on active as well as inactive compounds, to create a probabilistic QSAR model, which is subsequently used to select lead compounds. The Binary QSAR method is based upon three characteristic properties of compounds that form a “descriptor” of the likelihood that a particular compound will or will not perform a required function: partial charge, molar refractivity (bonding interactions), and log P (lipophilicity of molecule). Each atom has a surface area in the molecule and it has these three properties associated with it. All atoms of a compound having a partial charge in a certain range are determined and the surface areas (Van der Walls Surface Area descriptor) are summed. The binary QSAR models are then used to make activity models or ADMET models, which are used to build a combinatorial library. Accordingly, lead compounds identified in initial screens, can be used to expand the list of compounds being screened to thereby identify highly active compounds.

### Nucleic Acid Aptamers

In another example, a binding molecule is a nucleic acid aptamer (adaptable oligomer). Aptamers are single stranded oligonucleotides or oligonucleotide analogs that are capable of forming a secondary and/or tertiary structure that provides the ability to bind to a particular target molecule, such as a protein or a small molecule, e.g., IL-3Rα. Thus, aptamers are the oligonucleotide analogy to antibodies. In general, aptamers comprise about 15 to about 100 nucleotides, such as about 15 to about 40 nucleotides, for example about 20 to about 40 nucleotides, since oligonucleotides of a length that falls within these ranges can be prepared by conventional techniques.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to an epitope of IL-3Rα) is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990; U.S. Pat. No. 5,270,163; and/or U.S. Pat. No. 5,475,096.

Selection of Compounds that Specifically Bind to IL-3Rα

Suitable methods for selecting a compound (e.g., an antibody or antigen binding fragment thereof) that specifically binds to IL-3Rα, or an epitope thereof, are available to those skilled in the art.

For example, a screen may be conducted to identify compounds capable of binding to IL-3Rα. Any antibodies or antigen binding fragments that bind to IL-3Rα are then screened to identify those that bind to the epitope of interest using a method described herein.

For example, a phage display library displaying antibody fragments is screened with IL-3Rα or the N-terminal domain thereof or a soluble form thereof to identify proteins that bind thereto. Mutant forms of IL-3Rα (e.g., comprising alanine point mutations as described herein) to which the antibody fragment is not to be able to detectably bind are then used to remove cross-reactive proteins. A screening process for immunization of a non-human mammal can also be devised based on the foregoing as can a screening method for identifying other compounds described herein.

In a further example, IL-3Rα or a cell expressing same or the N-terminal domain thereof or a soluble form thereof is contacted with antibody 7G3. A library (e.g., a phage display library) is then brought into contact with the IL-3Rα or a cell expressing same or the N-terminal domain thereof or a soluble form thereof and compounds (or phage or cells expressing compounds) that can compete with 7G3 for binding selected.

In a still further example, a chimeric protein comprising, e.g., a mouse IL-3Rα in which an epitope of interest from a human IL-3Rα is substituted for the corresponding mouse sequence. This chimeric protein is then used to immunize mice (which are less likely to induce an immune response against the mouse protein) and/or to screen a library. The resulting compounds (e.g., antibodies) are then screened to identify those that bind to IL-3Rα (particularly at an epitope of interest as described herein) and not mouse IL-3Rα.

In one example, a compound of the disclosure contacts one or more residues in IL-3Rα as described herein. In one example, the compound contacts at least residues corresponding to amino acids 51 and 59 of SEQ ID NO: 1. Methods for determining if a compound contacts a residue will be apparent to the skilled artisan. For example, residues contacted by a compound can be determined by X-ray crystallographic analysis or modeling of the compound bound to IL-3Rα or a region thereof (e.g., the extracellular domain) or by mutation analysis, e.g., alanine scanning mutagenesis (for example, as described and/or exemplified herein). For example, mutant forms of IL-3Rα or a region thereof (e.g., the extra cellular domain) and in which one or more (e.g., two or three) residues are mutated to alanine (or a conservative amino acid substitution) are produced and binding of the mutant forms to the compound determined. Mutations that reduce binding to the compound by more than a threshold or that reduce binding to the greatest degree compared to other mutations are considered to be involved in binding to the compound.

### Constant Regions

The present disclosure encompasses compounds (e.g., antibodies and antigen binding fragments thereof) comprising a constant region of an antibody and/or a Fc region of an antibody.

Sequences of constant regions and/or Fc regions useful for producing the immunoglobulins, antibodies or antigen binding fragments of the present disclosure may be obtained from a number of different sources. In some examples, the constant region, Fc or portion thereof of the compound is derived from a human antibody. The constant region, Fc or portion thereof may be derived from any antibody class, including IgA, IgM, IgG, IgD, IgA and IgE, and any antibody isotype, including IgG1, IgG2, IgG3 and IgG4. In one example, the constant region or Fc is human isotype IgG1 or human isotype IgG2 or human isotype IgG3 or a hybrid of any of the foregoing.

In one example, the constant region or Fc region is capable of inducing an effector function. For example, the constant region or Fc region is a human IgG1 or IgG3 Fc region. In another example, the constant region or Fc region is a hybrid of an IgG1 and an IgG2 constant region or Fc region or a hybrid of an IgG1 and an IgG3 constant region or Fc region or a hybrid of an IgG2 and an IgG3 constant region or Fc region. Exemplary hybrids of human IgG1 and IgG2 constant region or Fc regions are described in Chappel et al., Proc. Natl Acad. Sci. USA, 88: 9036-9040, 1991.

Methods for determining whether or not a Fc region can induce effector function will be apparent to the skilled artisan and/or described herein.

### Effector Function

Suitably, a compound of the disclosure (e.g., an anti-IL-3Rα antibody or antigen binding fragment thereof) has or displays an effector function that facilitates or enables at least partial depletion, substantial depletion or elimination of IL-3Rα expressing cells. Such an effector function may be enhanced binding affinity to Fc receptors, antibody-dependent cell-mediated cytotoxicity (ADCC), antibody-dependent cell mediated phagocytosis (ADCP) and/or complement dependent cytotoxicity (CDC).

As will be apparent to the skilled artisan based on the description herein, some examples of the present disclosure include a compound (e.g., an antibody or antigen binding fragment thereof) capable of inducing effector function.

For the IgG class of antibodies, some effector functions (e.g., ADCC and ADCP) are governed by engagement of the Fc region with a family of receptors referred to as the Fcγ receptors (FcγRs) which are expressed on a variety of immune cells and/or with complement, e.g., C1q (e.g., CDC).

Formation of the Fc/FcγR complex recruits immune cells to sites of bound antigen, typically resulting in signaling and subsequent immune responses. Methods for optimizing the binding affinity of the FcγRs to the antibody Fc region in order to enhance the effector functions, e.g., to alter the ADCC activity relative to the “parent” Fc region, are known to persons skilled in the art. These methods can include modification of the Fc region of the antibody to enhance its interaction with relevant Fc receptors and increase its potential to facilitate ADCC and ADCP. Enhancements in ADCC activity have also been described following the modification of the oligosaccharide covalently attached to IgG1 antibodies at the conserved Asn297 in the Fc region.

It will be appreciated by the skilled artisan that in some non-limiting examples, enhancing effector function such as ADCC may be achieved by modification of a compound (e.g., an antibody) which has a normally glycosylated wild-type constant domain, including alteration or removal of glycosylation (see for example WO00/61739) and/or amino acid sequence mutations (see for example WO2008036688).

In one example, the compound binds to IL-3Rα in such a manner that it is capable of inducing an effector function, such as, ADCC.

In one example, the compound binds to an epitope within IL-3Rα that permits it to induce an effector function, such as ADCC.

In another example, the compound is capable of binding to IL-3Rα on a cell in a mammal to thereby induce an effector function, such as ADCC.

For example, the compound remains bound to IL-3Rα on the surface of a cell for a time sufficient to induce an effector function, such as ADCC. For example, the compound is not internalized too quickly to permit ADCC to be induced.

Alternatively, or in addition, the compound is bound to the IL-3Rα on the surface of the cell in a manner permitting an immune effector cell to bind to a constant region or Fc region in the compound and induce an effector function, such as ADCC. For example, the Fc region of the compound is exposed in such a manner when the compound is bound to the IL-3Rα that is capable of interacting with a Fc receptor (e.g., a FcγR) on an immune effector cell. In the context of the present disclosure, the term “immune effector cell” shall be understood to mean any cell that expresses a Fc receptor and that is capable of killing a cell to which it is bound by ADCC or ADCP. In one example, the immune effector cell is a NK cell.

Each of the above paragraphs relating to effector functions of an antibody or antigen binding fragment shall be taken to apply mutatis mutandis to inducing CDC. For example, the compound is bound to the IL-3Rα on the surface of the cell in a manner permitting complement component C1q to bind to a constant region or Fc region in the compound and induce CDC.

Moreover, each of the above paragraphs relating to effector functions of an antibody or antigen binding fragment shall be taken to apply mutatis mutandis to inducing cell-mediated effector function (e., ADCC and/or ADCP) by virtue of a compound other than a Fc region or constant region of an antibody. For example, the cell-mediated effector function is elicited using a compound that binds to IL-3Rα as described herein and to an immune effector cells (e.g., by virtue of binding to CD19 on NK cells and/or CD4 on T cells).

In one example, the compound is capable of inducing an enhanced level of effector function.

In one example, the level of effector function induced by the constant region or Fc region is enhanced relative to a wild-type constant region or Fc region of an IgG1 antibody or a wild-type constant region or Fc region of an IgG3 antibody.

In another example, the constant region or Fc region is modified to increase the level of effector function it is capable of inducing compared to the constant region or Fc region without the modification. Such modifications can be at the amino acid level and/or the secondary structural level and/or the tertiary structural level and/or to the glycosylation of the constant region or Fc region.

The skilled addressee will appreciate that greater effector function may be manifested in any of a number of ways, for example as a greater level of effect, a more sustained effect or a faster rate of effect.

In one example, the constant region or Fc region comprises one or more amino acid modifications that increase its ability to induce enhanced effector function. In one example, the constant region or Fc region binds with greater affinity to one or more FcγRs. In one example, the constant region or Fc region has an affinity for an FcγR that is more than 1-fold greater than that of a wild-type constant region or Fc region or more than 5-fold greater than that of a wild-type constant region or Fc region or between 5-fold and 300-fold greater than that of a wild-type constant region or Fc region. In one example, the constant region or Fc region comprises at least one amino acid substitution at a position selected from the group consisting of: 230, 233, 234, 235, 239, 240, 243, 264, 266, 272, 274, 275, 276, 278, 302, 318, 324, 325, 326, 328, 330, 332, and 335, numbered according to the EU index of Kabat. In one example, the constant region or Fc region comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y, numbered according to the EU index of Kabat. In one example, the constant region or Fc region comprises amino acid substitutions selected from the group consisting of V264I, F243L/V264I, L328M, I332E, L328M/I332E, V264I/I332E, S298A/I332E, S239E/I332E, S239Q/I332E, S239E, A330Y, I332D, L328I/I332E, L328Q/I332E, V264T, V240I, V266I, S239D, S239D/I332D, S239D/I332E, S239D/I332N, S239D/I332Q, S239E/I332D, S239E/I332N, S239E/I332Q, S239N/I332D, S239N/I332E, S239Q/I332D, A330Y/I332E, V264I/A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L328I/I332E, S239E/V264I/I332E, S239Q/V264I/I332E, S239E/V264I/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, S324D, S324I, S324V, K326I, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T, S239D/A330Y/I332E/K326E, and S239D/A330Y/I332E/K326T, numbered according to the EU index of Kabat.

In another example, the constant region or Fc region binds to FcγRIIIa more efficiently than to FcγRIIb. For example, the constant region or Fc region comprises at least one amino acid substitution at a position selected from the group consisting of: 234, 235, 239, 240, 264, 296, 330, and 1332, numbered according to the EU index of Kabat. In one example, the constant region or Fc region comprises at least one amino acid substitution selected from the group consisting of: L234Y, L234I, L235I, S239D, S239E, S239N, S239Q, V240A, V240M, V264I, V264Y, Y296Q, A330L, A330Y, A330I, I332D, and I332E, numbered according to the EU index of Kabat. For example, the constant region or Fc region comprises amino acid substitutions selected from the group consisting of: I332E, V264I/I332E, S239E/I332E, S239Q/I332E, Y296Q, A330L, A330Y, I332D, S239D, S239D/I332E, A330Y/I332E, V264I/A330Y/I332E, A330L/I332E, V264I/A330L/I332E, L234Y, L234I, L235I, V240A, V240M, V264Y, A330I, S239D/A330L/I332E, S239D/S298A/I332E, S239N/S298A/I332E, S239D/V264I/I332E, S239D/V264I/S298A/I332E, and S239D/V264I/A330L/I332E, numbered according to the EU index of Kabat.

In a further example, the constant region or Fc region induces ADCC at a level greater than that mediated by a wild-type constant region or Fc region. For example, the constant region or Fc region induces ADCC at a level that is more than 5-fold or between 5-fold and 1000-fold greater than that induced by a wild-type constant region or Fc region. In one example, the constant region or Fc region comprises at least one amino acid substitution at a position selected from the group consisting of: 230, 233, 234, 235, 239, 240, 243, 264, 266, 272, 274, 275, 276, 278, 302, 318, 324, 325, 326, 328, 330, 332, and 335, numbered according to the EU index of Kabat. In one example, the constant region or Fc region comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y, numbered according to the EU index of Kabat. In one example, the constant region or Fc region comprises amino acid substitutions selected from the group consisting of: V264I, F243L/V264I, L328M, I332E, L328M/I332E, V264I/I332E, S298A/I332E, S239E/I332E, S239Q/I332E, S239E, A330Y, I332D, L328I/I332E, L328Q/I332E, V264T, V240I, V266I, S239D, S239D/I332D, S239D/I332E, S239D/I332N, S239D/I332Q, S239E/I332D, S239E/I332N, S239E/I332Q, S239N/I332D, S239N/I332E, S239Q/I332D, A330Y/I332E, V264I/A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L328I/I332E, S239E/V264I/I332E, S239Q/V264I/I332E, S239E/V264I/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, S324D, S324I, S324V, K326I, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T, S239D/A330Y/I332E/K326E, and S239D/A330Y/I332E/K326T, numbered according to the EU index of Kabat.

In one example, the constant region or Fc region comprises the following amino acid substitutions S239D/I332E, numbered according to the EU index of Kabat. This constant region or Fc region has about 14 fold increase in affinity for FcγRIIIa compared to a wild-type constant region or Fc region and about 3.3 increased ability to induce ADCC compared to a wild-type constant region or Fc region. In one example, the constant region comprises a sequence set forth between residues 121-450 (inclusive) of SEQ ID NO: 13. In one example, the Fc region comprises a sequence set forth between residues 234-450 of SEQ ID NO: 13.

In one example, the constant region or Fc region comprises the following amino acid substitutions S239D/A330L/I332E, numbered according to the EU index of Kabat. This constant region or Fc region has about 138 fold increase in affinity for FcγRIIIa compared to a wild-type constant region or Fc region and about 323 increased ability to induce ADCC compared to a wild-type constant region or Fc region.

Additional amino acid substitutions that increase ability of a Fc region to induce effector function are known in the art and/or described, for example, in U.S. Pat. No. 6,737,056 or U.S. Pat. No. 7,317,091.

In one example, the glycosylation of the constant region or Fc region is altered to increase its ability to induce enhanced effector function. In this regard, native antibodies produced by mammalian cells typically comprise a branched, biantennary oligosaccharide that is generally attached by an N-linkage to Asn297 of the C_{H}2 domain of the constant region or Fc region. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some examples, constant regions or Fc regions according to the present disclosure comprise a carbohydrate structure that lacks fucose attached (directly or indirectly) to an Fc region, i.e., the Fc region is “afucosylated”. Such variants may have an improved ability to induce ADCC. Methods for producing afucosylated Fc regions or constant regions include, expressing the immunoglobulin or antibody in a cell line incapable of expressing α-1,6-fucosyltransferase (FUT8) (e.g., as described in Yumane-Ohnuki et al., Biotechnol. Bioengineer., 87: 614-622, 2004), expressing the immunoglobulin or antibody in cells expressing a small interfering RNA against FUT8 (e.g., as described in Mori et al., Biotechnol. Bioengineer., 88: 901-908, 2004), expressing the antibody or antigen binding fragment in cells incapable of expressing guanosine diphosphate (GDP)-mannose 4,6-dehydratase (GMD) (e.g., as described in Kanda et al., J. Biotechnol., 130: 300-310, 2007). The present disclosure also contemplates the use of compounds having a reduced level of fucosylation, e.g., produced using a cell line modified to express β-(1,4)-N-acetylglucosaminyltransferase III (GnT-III) (e.g., as described in Umāna et al., Nat. Biotechnol., 17: 176-180, 1999).

In one example, an antibody or antigen binding fragment according to the present disclosure is afucosylated. For example, the immunoglobulin or antibody is produced in a cell (e.g., a mammalian cell, such as a CHO cell) that does not express FUT8.

Other methods include the use of cell lines which inherently produce Fc regions or constant regions or antigen binding fragments capable of inducing enhanced Fc-mediated effector function (e.g. duck embryonic derived stem cells for the production of viral vaccines, WO2008/129058; Recombinant protein production in avian EBX® cells, WO2008/142124).

Compounds (e.g., antibodies or antigen binding fragments) useful in the methods of the present disclosure also include those with bisected oligosaccharides, e.g., in which a biantennary oligosaccharide attached to the constant region or Fc region is bisected by GlcNAc. Such compounds may have reduced fucosylation and/or improved ADCC function. Examples of such compounds are described, e.g., in U.S. Pat. No. 6,602,684 and US20050123546.

Compounds (e.g., antibodies or antigen binding fragments) with at least one galactose residue in the oligosaccharide attached to the constant region or Fc region are also contemplated. Such antibodies or antigen binding fragments may have improved CDC function. Such immunoglobulins are described, e.g., in WO1997/30087 and WO1999/22764.

Methods for determining the ability of a compound to induce effector function and known in the art and/or described in more detail herein.

### Additional Modifications

The present disclosure also contemplates additional modifications to constant regions or Fc regions of compounds (e.g., antibodies or antigen binding fragments).

For example, constant region of Fc region comprises one or more amino acid substitutions that increase the half-life of the antibody or fragment. For example, the constant region or Fc region comprises one or more amino acid substitutions that increase the affinity of the constant region or Fc region for the neonatal Fc region (FcRn). For example, the constant region or Fc region has increased affinity for FcRn at lower pH, e.g., about pH 6.0, to facilitate Fc/FcRn binding in an endosome. In one example, the constant region or Fc region has increased affinity for FcRn at about pH 6 compared to its affinity at about pH 7.4, which facilitates the re-release of constant region or Fc into blood following cellular recycling. These amino acid substitutions are useful for extending the half life of a Fc containing or constant region containing compound, by reducing clearance from the blood.

Exemplary amino acid substitutions include T250Q and/or M428L according to the EU numbering system of Kabat. Additional or alternative amino acid substitutions are described, for example, in US20070135620.

### Protein Production

### Recombinant Expression

In one example, a compound as described herein is a peptide or polypeptide (e.g., is an antibody or antigen binding fragment thereof). In one example, the compound is recombinant.

In the case of a recombinant peptide or polypeptide, nucleic acid encoding same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce immunoglobulin or antibody protein.

Exemplary cells used for expressing a peptide or polypeptide are CHO cells, myeloma cells or HEK cells. The cell may further comprise one or more genetic mutations and/or deletions that facilitate expression of a peptide or polypeptide (e.g., antibody or antigen binding fragment thereof). One non-limiting example is a deletion of a gene encoding an enzyme required for fucosylation of an expressed peptide or polypeptide (e.g., comprising a Fc region of an antibody). For example, the deleted gene encodes FUT8. A commercially available source of FUT8-deleted CHO cells is Biowa (Potelligent™ cells). For example, the cells used for expression of an afucosylated peptide or polypeptide are FUT8-deleted CHO cells, such as, Biowa's Potelligent™ cells.

Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells. Thus, another example of the disclosure provides an expression construct that comprises an isolated nucleic acid of the disclosure and one or more additional nucleotide sequences. Suitably, the expression construct is in the form of, or comprises genetic components of, a plasmid, bacteriophage, a cosmid, a yeast or bacterial artificial chromosome as are understood in the art. Expression constructs may be suitable for maintenance and propagation of the isolated nucleic acid in bacteria or other host cells, for manipulation by recombinant DNA technology and/or for expression of the nucleic acid or a compound of the disclosure.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding the compound (e.g., derived from the information provided herein), an enhancer element, a promoter, and a transcription termination sequence. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (Ula and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin or antibody promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

The host cells used to produce the compound (e.g., antibody or antigen binding fragment) may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for purifying a peptide or polypeptide (e.g., an antibody or antigen binding fragment) are known in the art and/or described herein.

Where a peptide or polypeptide is secreted into the medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The peptide or polypeptide prepared from cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbour Laboratory, (1988).

### Peptide Synthesis

A peptide is synthesized using a chemical method known to the skilled artisan. For example, synthetic peptides are prepared using known techniques of solid phase, liquid phase, or peptide condensation, or any combination thereof, and can include natural and/or unnatural amino acids. Amino acids used for peptide synthesis may be standard Boc (Nα-amino protected Nα-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Na-amino protected amino acids can be obtained from various commercial sources, such as, for example, Fluka, Bachem, Advanced Chemtech, Sigma, Cambridge Research Biochemical, Bachem, or Peninsula Labs.

Generally, chemical synthesis methods comprise the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do notracemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis. Synthesis. Biology, Vol. 1, for classical solution synthesis. These methods are suitable for synthesis of a peptide of the present disclosure.

A peptide as described herein can also be chemically prepared by other methods such as by the method of simultaneous multiple peptide synthesis. See, e. g., Houghten Proc. Natl. Acad. Sci. USA 82: 5131-5135, 1985 or U.S. Pat. No. 4,631,211.

### Nucleic Acid Synthesis

Methods for producing/synthesizing nucleic acid-based compounds of the disclosure |are known in the art. For example, oligonucleotide synthesis is described, in Gait (editor) Oligonucleotide Synthesis: A Practical Approach, IRL Press, Oxford (1984). For example, a probe or primer may be obtained by biological synthesis (e.g. by digestion of a nucleic acid with a restriction endonuclease) or by chemical synthesis. For short sequences (up to about 100 nucleotides) chemical synthesis is desirable.

For longer sequences standard replication methods employed in molecular biology are useful, such as, for example, the use of M13 for single stranded DNA as described by Messing Methods Enzymol, 101: 20-78, 1983.

Other methods for oligonucleotide synthesis include, for example, phosphotriester and phosphodiester methods (Narang, editor, “Synthesis and Applications of DNA and RNA” Academic Press, New York (1987)) and synthesis on a support (Beaucage, et al., Tetrahedron Letters, 22: 1859-1862, 1981) as well as phosphoramidate technique, Caruthers, M. H., et al., “Methods in Enzymology,” Vol. 154, pp. 287-314 (1988), and others described in Narang (1987), and the references contained therein.

### Assaying Activity of Compound

Compounds of the disclosure are readily screened for biological activity, e.g., as described below.

### Binding Assays

One form of such an assay is an antigen binding assay, e.g., as described in Scopes (In: Protein purification: principles and practice, Third Edition, Springer Verlag, 1994). Such a method generally involves labeling the compound (e.g., an antibody or antigen binding fragment) and contacting it with immobilized antigen. Following washing to remove non-specific bound protein, the amount of label and, as a consequence, bound compound is detected. Of course, the compound can be immobilized and the antigen labeled. Panning-type assays, e.g., as described herein can also be used.

### Epitope Mapping

In another example, the epitope bound by a compound (e.g., an antibody or antigen binding fragment) described herein is mapped. Epitope mapping methods will be apparent to the skilled artisan. For example, a series of overlapping peptides spanning the IL-3Rα sequence or a region thereof comprising an epitope of interest, e.g., peptides comprising 20-50 amino acids are produced. The compound is then contacted to each peptide and the peptide(s) to which it binds determined. This permits determination of peptide(s) comprising the epitope to which the compound binds. If multiple non-contiguous peptides are bound by the protein, the protein may bind a conformational epitope.

Alternatively, or in addition, amino acid residues within IL-3Rα are mutated, e.g., by alanine scanning mutagenesis or substitution with conservative amino acid changes (e.g., as exemplified herein), and mutations that reduce or prevent binding of the compound are determined. Any mutation that reduces or prevents binding of the compound is likely to be within the epitope bound by the compound. Exemplary alanine scanning mutagenesis methods are exemplified herein.

A further method involves binding IL-3Rα or a region thereof to an immobilized compound of the present disclosure and digesting the resulting complex with proteases. Peptide that remains bound to the immobilized compound are then isolated and analyzed, e.g., using mass spectrometry, to determine their sequence.

A further method involves converting hydrogens in IL-3Rα or a region thereof to deutrons and binding the resulting protein to an immobilized compound of the present disclosure. The deutrons are then converted back to hydrogen, the IL-3Rα or region thereof isolated, digested with enzymes and analyzed, e.g., using mass spectrometry to identify those regions comprising deutrons, which would have been protected from conversion to hydrogen by the binding of the antibody or antigen binding fragment.

### Determining Competitive Binding

Assays for determining a compound that competitively inhibits binding of an antibody described herein will be apparent to the skilled artisan. For example, the antibody is conjugated to a detectable label, e.g., a fluorescent label or a radioactive label. The labeled antibody and the compound are then mixed and contacted with IL-3Rα or a region thereof or a cell expressing same. The level of bound labeled antibody is then determined and compared to the level determined when the labeled antibody is contacted with the IL-3Rα, region or cells in the absence of the compound. If the level of labeled antibody is reduced in the presence of the compound compared to the absence of the compound, the compound is considered to competitively inhibit binding of the labeled antibody to IL-3Rα.

In another example, the compound is permitted to bind to IL-3Rα or a region thereof or a cell expressing same prior to contacting the IL-3Rα, region or cell with the labeled antibody. A reduction in the amount of bound labeled antibody in the presence of the compound compared to in the absence of the compound indicates that the compound competitively inhibits binding of the labeled antibody to IL-3Rα. More specifically, a soluble form of IL-3Rα or a cell expressing IL-3Rα is contacted with a compound of the disclosure for a time and under conditions to permit binding. The IL-3Rα or cell is then contacted with labeled 7G3 or CSL362 for a time an under conditions sufficient to permit binding. Following washing to remove unbound 7G3 or CSL362, the level of bound label is determined, e.g., by FACS or Surface Plasmon Resonance. A compound that reduces the level fo bound label compared to the level fo label detected following contacting soluble IL-3Rα or a cell expressing IL-3Rα in the with labeled 7G3 or CSL362 absence of compound competitively inhibits binding of 7G3 or CSL362. A reciprocal assay can also be performed using a labeled test compound.

Any of the foregoing assays can be performed with a mutant form of IL-3Rα and/or SEQ ID NO: 1, e.g., as described herein.

### Determining Neutralization

In some examples of the present disclosure, a compound of the present disclosure.

Various assays are known in the art for assessing the ability of a compound to neutralize signaling of a ligand through a receptor.

In one example, the compound reduces or prevents IL-3 binding to the 3Rα chain and/or a heterodimer of IL-3Rα chain and IL-3Rβ chain. These assays can be performed as a competitive binding assay using labeled IL-3 and/or labeled compound. For example, labeled IL-3Rα or an extracellular region thereof fused to an Fc region of an antibody or a cell expressing IL-3R is immobilized and labeled IL-3 is then contacted to the immobilized receptor or cell in the presence or absence of a compound and the amount of bound label detected. A reduction in the amount of bound label in the presence of the compound compared to in the absence of the compound indicates that the compound reduces or prevents binding of IL-3 to IL-3R. By testing multiple concentrations of the compound an IC₅₀ is determined, i.e., a concentration of the protein that reduces the amount of IL-3 that binds to IL-3R, or an EC₅₀ can be determined, i.e., a concentration of the protein that achieves 50% of the maximum inhibition of binding of IL-3 to IL-3R achieved by the compound.

In another example, the compound reduces or prevents IL-3-mediated histamine release from basophils. For example, low density leukocytes comprising basophils are incubated with IgE, IL-3 and various concentrations of the antibody or antigen binding fragment. Control cells do not comprise immunoglobulin (positive control) or IL-3 (negative control). The level of released histamine is then assessed using a standard technique, e.g., RIA. A compound that reduces the level of histamine release to a level less than the positive control is considered to neutralize IL-3 signaling. In one example, the level of reduction is correlated with compound concentration. An exemplary method for assessing IL-3-mediated histamine release is described, for example, in Lopez et al., J. Cell. Physiol., 145: 69, 1990.

In a further example, the compound reduces or prevents IL-3-mediated proliferation of leukemic cell line TF-1. For example, TF-1 cells are cultured without IL-3 or GM-CSF for a time sufficient for them to stop proliferating (e.g., 24-48 hours). Cells are then cultured in the presence of IL-3 and various concentrations of the compound. Control cells are not contacted with the compound (positive control) or IL-3 (negative control). Cell proliferation is then assessed using a standard technique, e.g., ³H-thymidine incorporation. A compound that reduces or prevents cell proliferation in the presence of IL-3 to a level less than the positive control is considered to neutralize IL-3 signaling.

Another assay for assessing IL-3 signaling neutralization comprises determining whether or not the compound reduces or prevents IL-3-mediated effects on endothelial cells. For example, human umbilical vein endothelial cells (HUVECs) are cultured in the presence of IL-3 (optionally, with IFN-γ) and various concentrations of the compound. The amount of secreted IL-6 is then assessed, e.g., using an enzyme linked immunosorbent assay (ELISA). Control cultures do not comprise the compound (positive control) or IL-3 (negative control). A compound that reduces or prevents IL-6 production in the presence of IL-3 to a level less than the positive control is considered to neutralize IL-3 signaling.

Other methods for assessing neutralization of IL-3 signaling are contemplated by the present disclosure.

### Determining Effector Function

Methods for assessing ADCC activity are known in the art.

In one example, the level of ADCC activity is assessed using a ⁵¹Cr release assay, a europium release assay or a ³⁵S release assay. In each of these assays, cells expressing IL-3Rα are cultured with one or more of the recited compounds for a time and under conditions sufficient for the compound to be taken up by the cell. In the case of a ³⁵S release assay, cells expressing IL-3Rα can be cultured with ³⁵S-labeled methionine and/or cysteine for a time sufficient for the labeled amino acids to be incorporated into newly synthesized proteins. Cells are then cultured in the presence or absence of a compound of the disclosure and in the presence of immune effector cells, e.g., peripheral blood mononuclear cells (PBMC) and/or NK cells. The amount of ⁵¹Cr, europium and/or ³⁵S in cell culture medium is then detected, and an increase in the presence of the compound compared to in the absence of the compound indicates that the antibody or antigen binding fragment has effector function. Exemplary publications disclosing assays for assessing the level of ADCC induced by a compound include Hellstrom, et al. Proc. Natl Acad. Sci. USA 83:7059-7063, 1986 and Bruggemann, et al., J. Exp. Med. 166:1351-1361, 1987.

Other assays for assessing the level of ADCC induced by a compound include ACTI™ nonradioactive cytotoxicity assay for flow cytometry (CellTechnology, Inc. CA, USA) or CytoTox 96® non-radioactive cytotoxicity assay (Promega, Wis., USA).

Alternatively, or additionally, effector function of a compound is assessed by determining its affinity for one or more FcγRs, e.g., as described in U.S. Pat. No. 7,317,091.

C1q binding assays may also be carried out to confirm that the compound is able to bind C1q and may induce CDC. To assess complement activation, a CDC assay may be performed (see, for example, Gazzano-Santoro et al, J. Immunol. Methods 202: 163, 1996.

### Determining Affinity

Optionally, the dissociation constant (Kd) or association constant (Ka) or equilibrium constant (K_{D}) of a compound for IL-3Rα or an epitope thereof is determined. These constants for a compound (e.g., an antibody or antigen binding fragment) are, in one example, measured by a radiolabeled or fluorescently-labeled IL-3Rα-binding assay. This assay equilibrates the compound with a minimal concentration of labeled IL-3Rα (or a soluble form thereof, e.g., comprising an extracellular region of IL-3Rα fused to an Fc region) in the presence of a titration series of unlabeled IL-3Rα. Following washing to remove unbound IL-3Rα, the amount of label is determined.

Affinity measurements can be determined by standard methodology for antibody reactions, for example, immunoassays, surface plasmon resonance (SPR) (Rich and Myszka Curr. Opin. Biotechnol 11::54, 2000; Englebienne Analyst. 123: 1599, 1998), isothermal titration calorimetry (ITC) or other kinetic interaction assays known in the art.

In one example, the constants are measured by using surface plasmon resonance assays, e.g., using BIAcore surface plasmon resonance (BIAcore, Inc., Piscataway, N.J.) with immobilized IL-3Rα or a region thereof. Exemplary SPR methods are described in U.S. Pat. No. 7,229,619.

### Assessing Therapeutic Efficacy

### In Vitro Assays

Various in vitro assays are available to assess the ability of a compound of the disclosure to treat a disease or condition described herein.

For example, a compound is assessed for its ability to kill a cell, e.g., a cancer cell, such as leukemic cell, using a method described herein.

In another example, immune cells, e.g., pDCs and/or basophils or cell populations comprising same (e.g., PBMC) are cultured in the presence or absence of a compound and an inducer of those cells that occurs in a disease or condition (e.g., CpG oligonucleotides and/or immune complexes). The efficacy of the compound in treating the disease or condition is then assessed, e.g., by determining the level of IFNα secreted into cell culture medium using an ELISA. Alternatively or in addition the level of histamine secretion or IL-4, IL-6 and/or IL-13 secretion is assessed. A reduction in the level of any of these cytokines compared to in the absence of the compound (or in the presence of an isotype control, e.g., in the case of an antibody) indicates that the antibody or antigen binding fragment is suitable for treating the disease or condition. Alternatively, or in addition, the level of cell death is assessed. An increase in cell death is indicative of a compound suitable for treating the disease or condition.

### In Vivo Assays

In one example, the efficacy of a compound to treat a disease or condition is assessed using an in vivo assay.

In one example, a xenotransplantation model of a cancer is used to assess therapeutic efficacy. For example, NOD/SCID mice are irradiated and optionally treated with anti-CD122 antibody to deplete NK cells. Human leukemic cells (e.g., acute myeloid leukemia cells) and mouse or human bone marrow stem cells are administered to the mice. Following cell engraftment, a compound is administered to the mice and the level of leukemic cells in circulation and/or bone marrow and/or lymph nodes is assessed. A reduction in the number of leukemic cells in circulation and/or bone marrow and/or lymph nodes in the presence of the compound compared to in the absence of the compound indicates therapeutic efficacy.

In another example, the compound is administered to a non-human animal (e.g., a non-human primate) and the number/level of immune cells, e.g., pDCs and/or basophils, in circulation is assessed. A compound that reduces the number/level of immune cells, e.g., pDCs and/or basophils compared to prior to administration and/or in a control mammal to which the compound has not been administered is considered suitable for treating the disease or condition.

In another example, the level of a cytokine, such as IFNα is detected in the circulation of a mammal, e.g., using an ELISA. A compound that reduces the level of the cytokine compared to the level prior to administration and/or in a control mammal to which the compound has not been administered is considered suitable for treating the disease or condition. Since cytokines such as IFNα are considered to play a role in some diseases/conditions, e.g., lupus.

### Compositions

Suitably, in compositions or methods for administration of the compound of the disclosure to a mammal, the compound is combined with a pharmaceutically acceptable carrier as is understood in the art. Accordingly, one example of the present disclosure provides a composition (e.g., a pharmaceutical composition) comprising the compound of the disclosure combined with a pharmaceutically acceptable carrier. In another example, the disclosure provides a kit comprising a pharmaceutically acceptable carrier suitable for combining or mixing with the compound prior to administration to the mammal. In this example, the kit may further comprise instructions for use.

In general terms, by “carrier” is meant a solid or liquid filler, binder, diluent, encapsulating substance, emulsifier, wetting agent, solvent, suspending agent, coating or lubricant that may be safely administered to any mammal, e.g., a human. Depending upon the particular route of administration, a variety of acceptable carriers, known in the art may be used, as for example described in Remington's Pharmaceutical Sciences (Mack Publishing Co. N.J. USA, 1991).

By way of example only, the carriers may be selected from a group including sugars (e.g. sucrose, maltose, trehalose, glucose), starches, cellulose and its derivatives, malt, gelatine, talc, calcium sulphate, oils inclusive of vegetable oils, synthetic oils and synthetic mono- or di-glycerides, lower alcohols, polyols, alginic acid, phosphate buffered solutions, lubricants such as sodium or magnesium stearate, isotonic saline and pyrogen-free water. For example, the carrier is compatible with, or suitable for, parenteral administration. Parenteral administration includes any route of administration that is not through the alimentary canal. Non-limiting examples of parenteral administration include injection, infusion and the like. By way of example, administration by injection includes intravenous, intra-arterial, intramuscular and subcutaneous injection. Also contemplated is delivery by a depot or slow-release formulation which may be delivered intradermally, intramuscularly and subcutaneously.

### Combination Therapies

In one example, the compound of the disclosure is administered in combination with another compound or therapeutic treatment useful for treating a disease or condition.

In one example, the compound is administered prior to, e.g., one month or one fortnight or one week prior to radiation therapy, e.g., for the treatment of cancer, such as a hematologic cancer, such as leukemia.

In one example, the other compound is a chemotherapy compound, such as caboplatin, cisplatin, cyclophosphamide, docetaxal, doxorubicin, erlotinib, etoposide, fluorouracil, irinotecan, methotrexate, paclitaxel, topotecan, vincristine or vinblastine. In one example, the chemotherapy compound is selected from the group consisting of methotrexate, l-asparaginase, vincristine, doxorubicin, danorubicin, cytarabine, idarubicin, mitoxantrone, cyclophosphamide, fludarabine, chlorambucil and combinations thereof.

In one example, the other compound is a chemotherapy compound used in the treatment of acute leukemia, such as, a compound selected from the group consisting of methotrexate, l-asparaginase, vincristine, doxorubicin, danorubicin, cytarabine, idarubicin, mitoxantrone and combinations thereof.

In one example, the other compound is a chemotherapy compound used in the treatment of acute lymphoblastic leukemia, such as, a compound selected from the group consisting of methotrexate, l-asparaginase, vincristine, doxorubicin, danorubicin and combinations thereof.

In a further example, the other compound is a chemotherapy compound such as azacytidine.

In one example, the other compound is a biologic useful for treating a cancer, e.g., rituximab, trastuzumab, bevacizumab, alemtuzumab, panitumumab, or cetuximab

In one example, the other compound is an anti-inflammatory compound. Alternatively, or additionally, the other compound is an immunosuppressant. Alternatively, or additionally, the other compound is a corticosteroid, such as prednisone and/or prednisolone. Alternatively, or additionally, the other compound is an antimalarial compound, such as hydroxychloroquine or chloroquinine. Alternatively, or additionally, the other compound is methotrexate. Alternatively, or additionally, the other compound is azathioprine. Alternatively, or additionally, the other compound is cyclophosphamide. Alternatively, or additionally, the other compound is mycophenolate mofetil. Alternatively, or additionally, the other compound is an anti-CD20 antibody (e.g., rituximab or ofatumumab). Alternatively, or additionally, the other compound is an anti-CD22 antibody (e.g., epratuzumab). Alternatively, or additionally, the other compound is an anti-TNF antibody (e.g., infliximab or adalimumab or golimumab). Alternatively, or additionally, the other compound is a CTLA-4 antagonist (e.g., abatacept, CTLA4-Ig). Alternatively, or additionally, the other compound is an anti-IL-6 antibody. Alternatively, or additionally, the other compound is a BLys antagonist, such as an anti-BLys antibody (e.g., belimumab).

### Dosages and Timing of Administration

For the prevention or treatment of a disease or condition or relapse thereof, the appropriate dosage of a compound active agent (e.g., an antibody or antigen binding fragment of the disclosure), will depend on the type of disease to be treated, the severity and course of the disease, whether the compound is administered for preventive or therapeutic purposes, previous therapy, the patient's clinical history and response to the compound, and the discretion of the attending physician. The particular dosage regimen, i.e., dose, timing, and repetition, will depend on the particular individual and that individual's medical history as assessed by a physician. Typically, a clinician will administer a compound until a dosage is reached that achieves the desired result.

Methods of the present disclosure are useful for treating, ameliorating or preventing the symptoms of diseases or conditions in a mammal, or for improving the prognosis of a mammal. Methods of the present disclosure are also useful for delaying development of or preventing diseases or condition in an individual at risk of developing the disease or condition or a relapse thereof.

For administration of the compounds described herein, normal dosage amounts may vary from about 10 ng/kg up to about 100 mg/kg of an individual's body weight or more per day. For repeated administrations over several days or longer, depending on the severity of the disease or disorder to be treated, the treatment can be sustained until a desired suppression of symptoms is achieved.

In some examples, the compound (e.g., a polypeptide based compound, such as an antibody or antigen binding fragment) is administered at an initial (or loading) dose of between about 1 mg/kg to about 30 mg/kg. The compound can then be administered at a maintenance dose of between about 0.0001 mg/kg to about 10 mg/kg. The maintenance doses may be administered every 7-30 days, such as, every 10-15 days, for example, every 10 or 11 or 12 or 13 or 14 or 15 days.

In the case of a mammal that is not adequately responding to treatment, multiple doses in a week may be administered. Alternatively, or in addition, increasing doses may be administered.

In another example, for mammals experiencing an adverse reaction, the initial (or loading) dose may be split over numerous days in one week or over numerous consecutive days.

Dosages for a particular compound may be determined empirically in mammals that have been given one or more administrations of the antibody or antigen binding fragment. To assess efficacy of a compound, a clinical symptom of a disease or condition can be monitored.

Administration of a compound according to the methods of the present disclosure can be continuous or intermittent, depending, for example, on the recipient's physiological condition, whether the purpose of the administration is therapeutic or prophylactic, and other factors known to skilled practitioners. The administration of a compound may be essentially continuous over a preselected period of time or may be in a series of spaced doses, e.g., either during or after development of a condition.

The present disclosure includes the following non-limiting examples.

### Example 1: Identification of a Neutralizing Epitope in IL-3Rα

### Domain of IL-3Rα Bound by Antibodies 7G3, 9F5 or 107D2

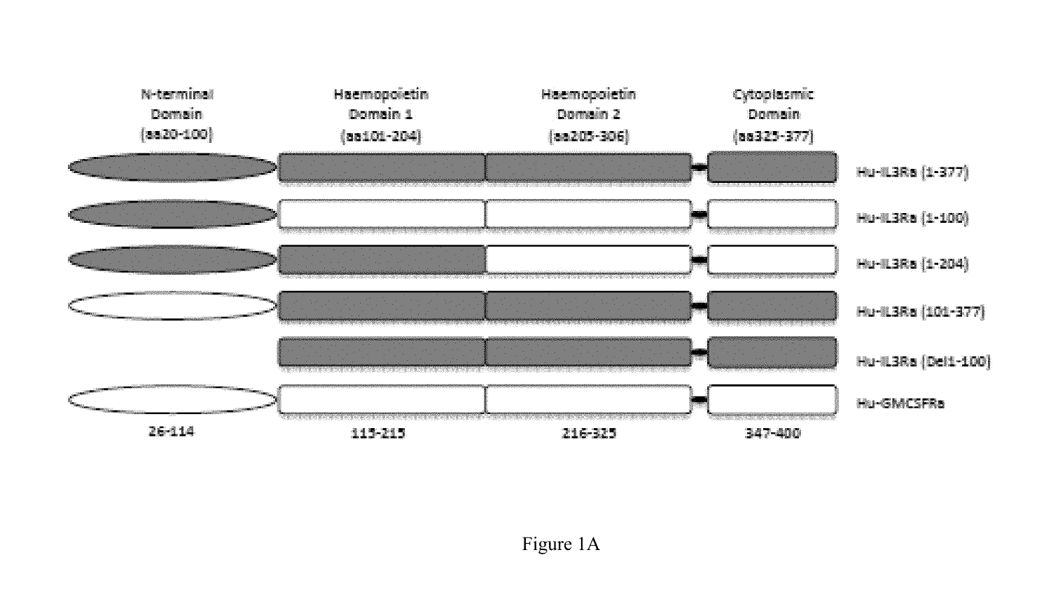

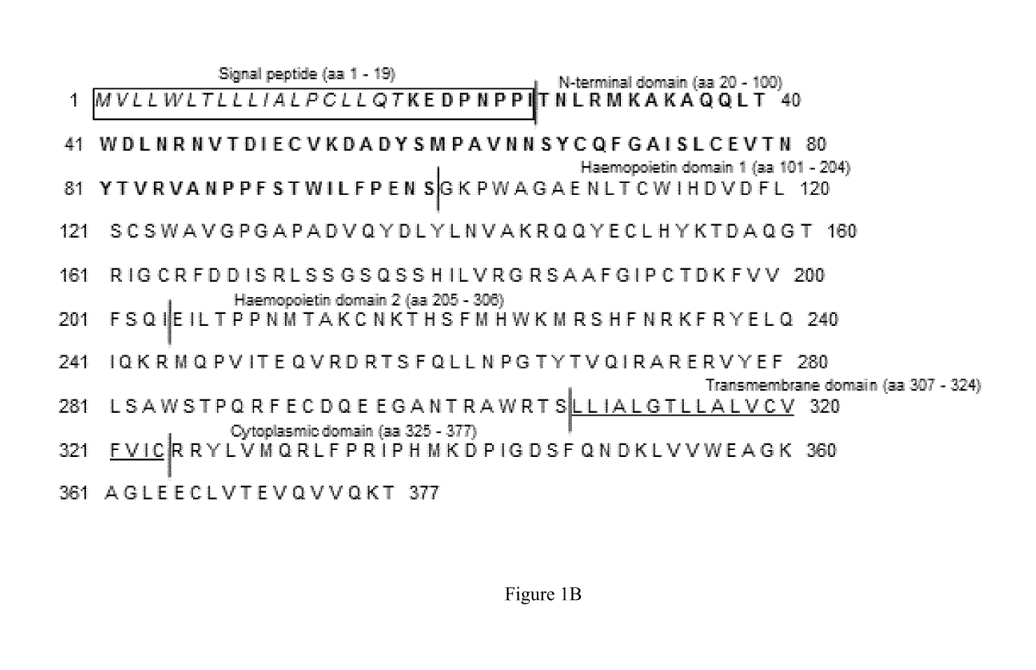

Expression constructs were generated of full length human IL-3Rα or of proteins comprising hIL-3Rα domains with various GM-CSFRα domains as depicted in FIG. 1A. The amino acid sequence of IL-3Rα showing each of the domains referred to in FIG. 1A is shown in FIG. 1B. 293F cells were transfected with expression constructs encoding the proteins depicted in FIG. 1A and stained with various anti-human IL-3Rα monoclonal antibodies (7G3, 9F5 or 107D2) and binding was detected by flow cytometry. Results are tabulated in Table 1 with positive staining (+) and negative staining (−) indicated.

TABLE 1
binding of 7G3, 9F5 and 107D2 to mutants of IL-3Rα

| mutant | 7G3-binding | 9F5-binding | 107D2-binding |
|---|---|---|---|
| 1-377 | + | + | + |
| 1-100 | + | + | − |
| 1-204 | + | + | − |
| 101-377 | − | − | + |
| del-100 | − | − | + |
| GM-CSFRa | − | − | − |

The data depicted in Table 1 indicate that antibodies 7G3 and 9F5 bind to an epitope within amino acids 1-100 (the N-terminal domain) of IL-3Rα, while antibody 107D2 binds to an epitope within residues 101-377 of IL-3Rα. Antibody 7G3 neutralizes IL-3 signaling, whereas antibodies 9F5 and 107D2 do not.

### Amino Acid Residues Involved in Binding of Antibodies 7G3, 9F5 or 107D2

Individual point mutants (either alanine or an evolutionary similar amino acid (also referred to as a conservative amino acid substitution) were generated for each amino acid in the N-terminal domain of human IL-3Rα. The human IL-3Rα point mutants were expressed in 293F cells and cells were stained with anti-IL-3Rα monoclonal antibodies (7G3, 9F5 or 107D2) and analyzed for cell surface binding by flow cytometry. Median fluorescence intensity (MFI) of each antibody was determined for wild type human IL-3Rα and for each IL-3Rα mutant. The MFI of wild type human IL-3Rα staining for each antibody was set as 100% then the MFI of each mutant was normalized to a percent of wild type staining. The normalized 7G3 staining was calculated as a percentage of normalized control antibody (9F5: dark gray bars or 107D2: light gray bars) staining according to the following equations:

1. mAb % wild type MFI=mutant MFI/wild type MFI×100/1.

2. 7G3% of control mAb=7G3% WT wild type/control mAb % wild type MFI×100/1.

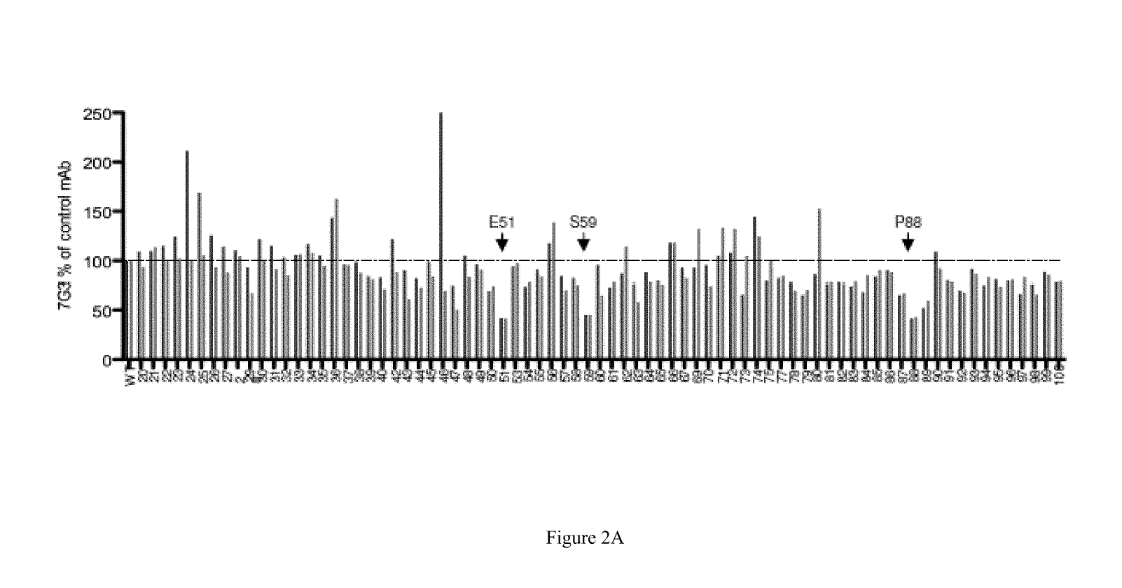

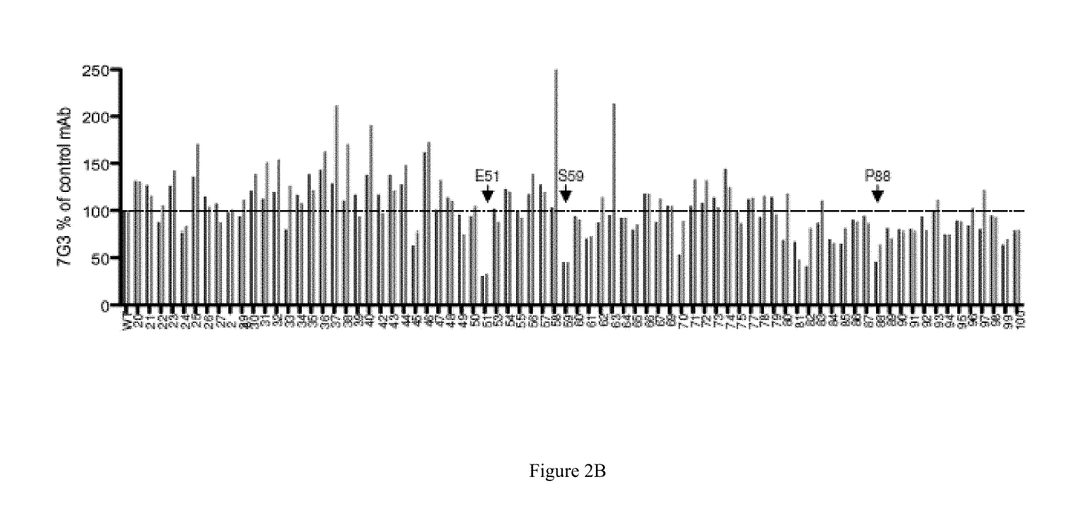

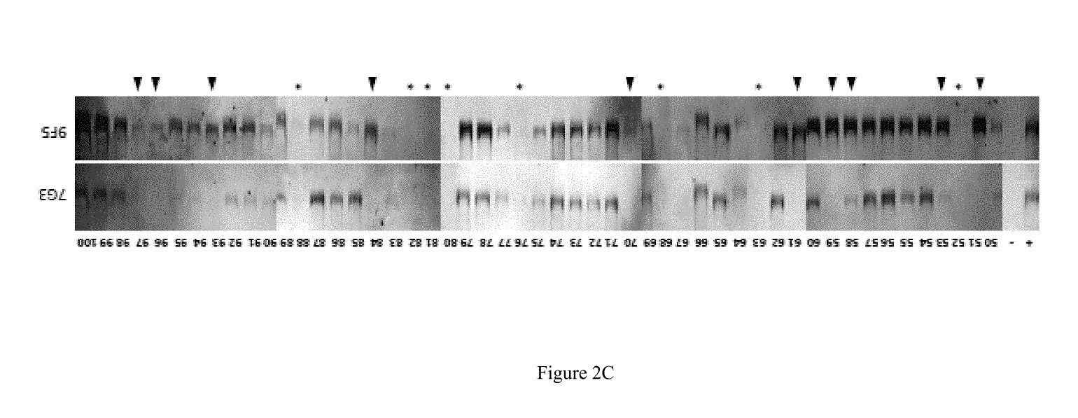

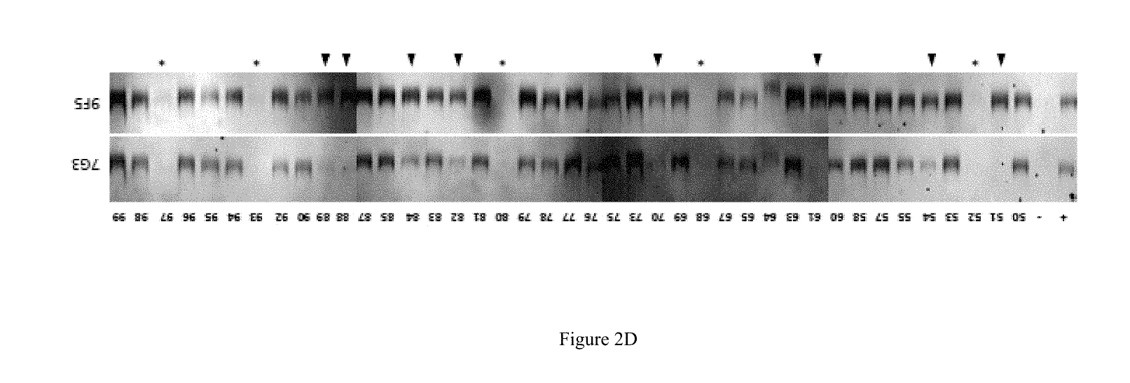

The experiment was repeated 3 times and representative histograms for alanine (FIG. 2A) and conservative (FIG. 2B) mutants are shown. A reduced level below the dotted line indicates a residue that is affects binding of 7G3 but not of 9F5 and/or 107D2)

The same alanine (FIG. 2C) and conservative (FIG. 2D) human IL-3Rα mutants were also tested for 7G3 and 9F5 binding by Western blot analysis following SDS-PAGE of whole cell extracts. Blots shown are representative. Triangles in FIGS. 2C and 2D indicate mutants with differential 7G3/9F3 binding.

### Amino Acid Residues Involved in Binding of Antibody CSL362

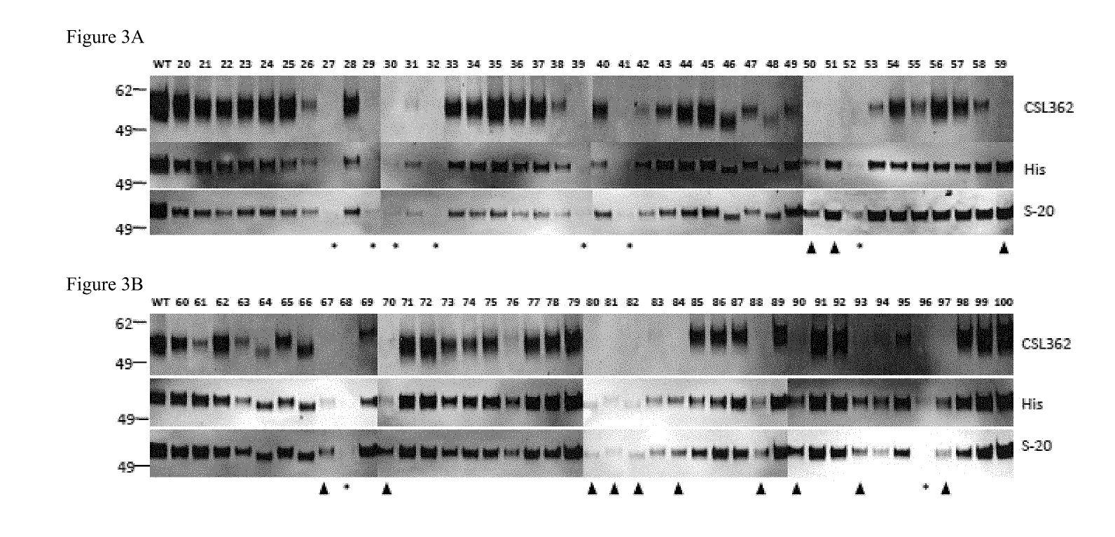

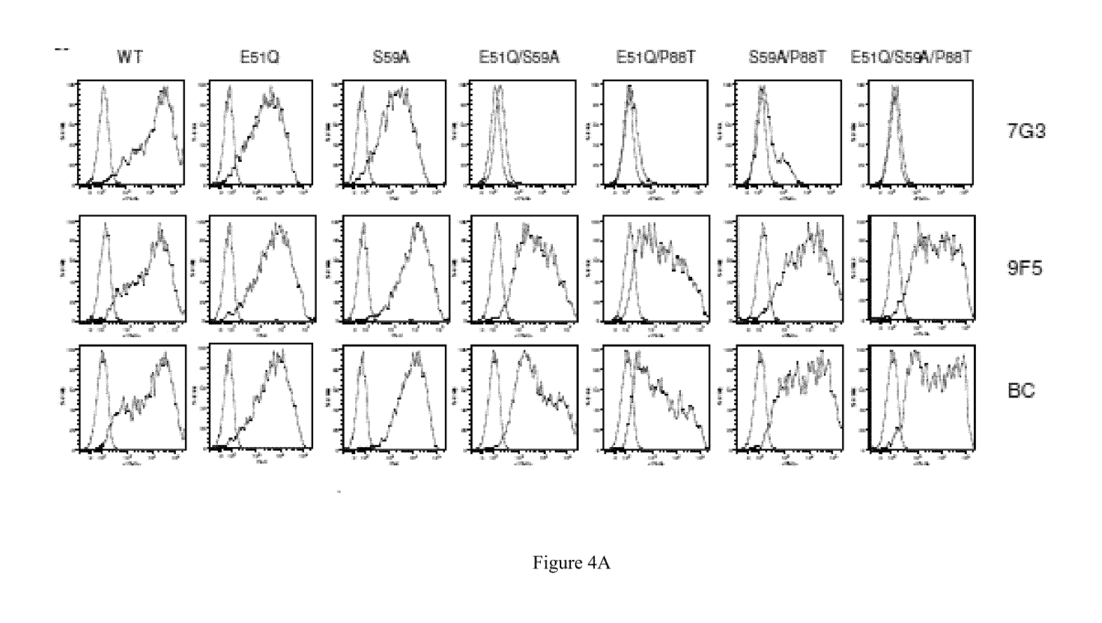

293FS cells were transiently transfected with expression plasmids encoding amino acids 1-306 of IL-3Rα with a C-terminal 6×His tag and alanine point mutants of 20-100. Supernatants were tested by Western blotting for binding to antibody CSL362 (comprising a V_{H} having a sequence set forth in SEQ ID NO: 4 and a V_{L} having a sequence set forth in SEQ ID NO: 5). Duplicate blots were also performed using an anti-His mAb and a control anti-IL-3Rα (S-20) Ab. FIG. 3 depicts results of representative blots, with triangles indicating mutants with differential CSL362 binding.

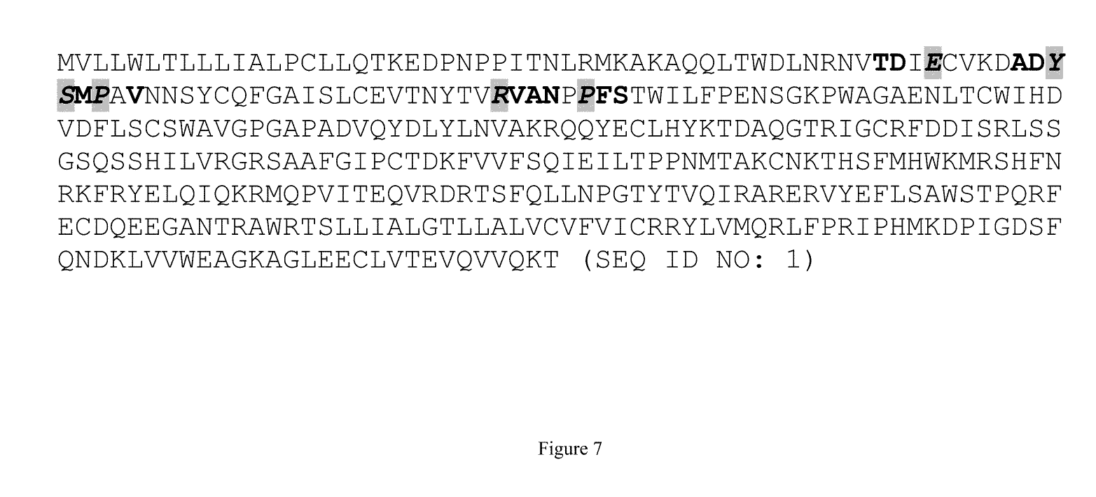

Table 2 and FIG. 7 shows a summary of the residues that, when mutated, resulted in reduced binding of either 7G3 and/or CSL362 as determined by Western Blotting.

TABLE 2
Summary of the residues that, when mutated, resulted in
reduced binding of either 7G3 and/or CSL362 as determined
by Western Blotting

| | | Cell surface | |
|---|---|---|---|
| | Cell surface | evolutionary | |
| | alanine | conserved | Soluble alanine |
| Amino acid | (7G3) | (7G3) | (CSL362) |
| I50 | + | + | − |
| E51 | − | − | − |
| V53 | − | + | +/− |
| K54 | + | − | + |
| Y58 | − | + | +/− |
| S59 | − | NA | − |
| P61 | − | − | +/− |
| Y67 | No expression | + | − |
| F70 | −* | − | − |
| N80 | No expression | No expression | −* |
| Y81 | No expression | + | −* |
| T82 | No expression | − | −* |
| R84 | − | − | − |
| P88 | No expression | − | − |
| P89 | + | − | + |
| F90 | +/−* | + | − |
| W93 | − | No expression | − |
| I94 | − | + | +/− |
| F96 | −* | + | No expression |
| P97 | −* | No expression | − |

Residues in italics are those that showed reduced binding across all Western blotting experiments. (+) denotes antibody binding, (−) denotes no antibody binding, (+/−) denotes weak antibody binding and (*) denotes poorly expressed IL-3Rα mutants.
Combinations of Mutations that Reduce Binding of Neutralizing Antibodies to IL-3Rα

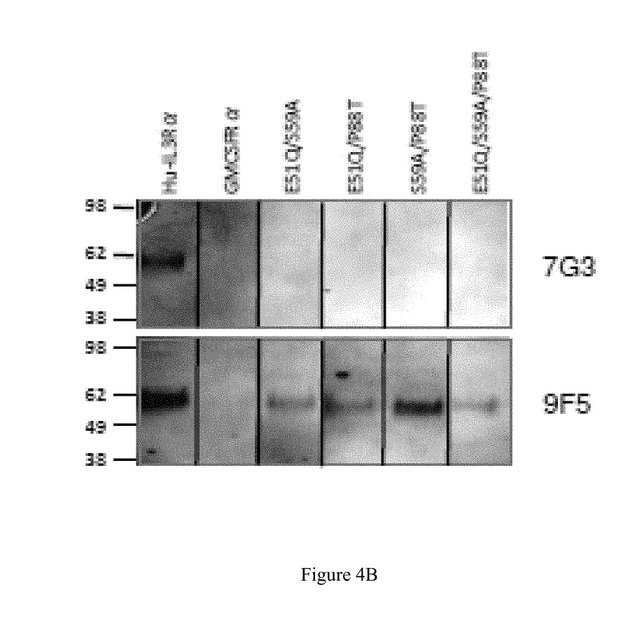

Cells expressing either double or triple combination point mutants (conservative amino acid substitutions) were generated for three residues (E51, S59 and P88) found to affect 7G3 binding to IL-3Rα. Cells expressing single point mutants for E51 and S59 were also made. FIG. 4A shows results of flow cytometry analysis of binding of antibodies (7G3, 9F5 or 107D2) to cells expressing wild type or mutant IL-3Rα. The data presented indicate that single point mutations at E51 or S59 reduced binding of 7G3 compared to wild type, while double or triple mutations reduced binding to the level observed in untransfected cells. These mutations had little effect on binding of non-neutralizing antibodies 9F5 and 107D2.

The same IL-3Rα mutants were also tested for 7G3 and 9F5 mAb binding by Western blot analysis following SDS-PAGE of whole cell extracts. Representative blots are shown in FIG. 4B. These data show that double or triple mutations in IL-3Rα reduced binding of 7G3 to undetectable levels, whereas 9F5 binding remained detectable.

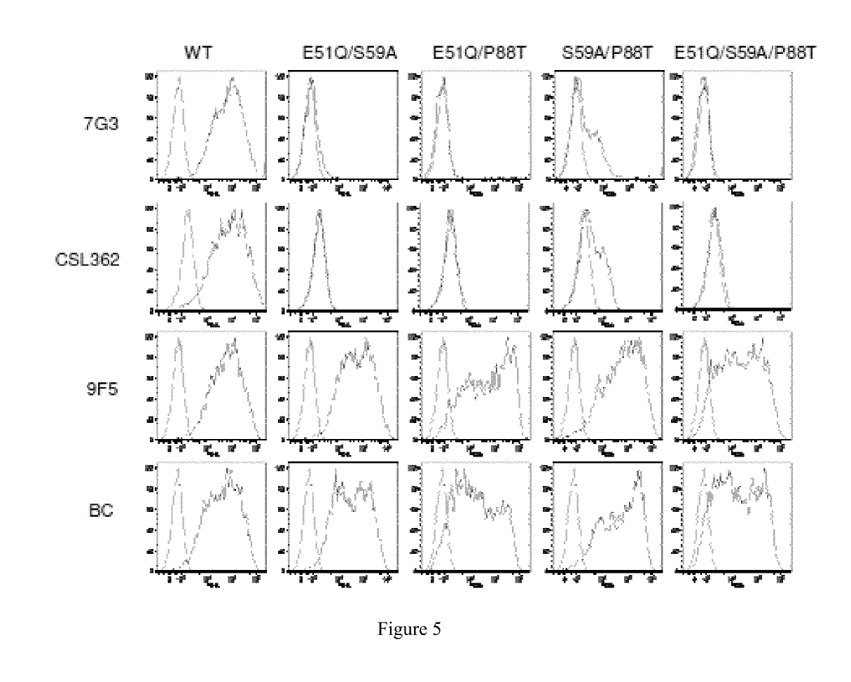

CSL362 was also tested for its ability to bind to cells expressing either double or triple combination point mutants as described above. FIG. 5 shows results of representative flow cytometry analysis of binding of antibodies (CSL362, 7G3, 9F5 or 107D2) to cells expressing wild type or mutant IL-3Rα. The data presented indicate that double or triple mutations reduced binding of 7G3 or CSL362 to the level observed in untransfected cells. These mutations had little effect on binding of non-neutralizing antibodies 9F5 and 107D2. These results are also summarized in Table 3.

TABLE 3
Binding of antibodies 7G3, CSL362, 9F5 and 107D2 to wild
type (WT) or mutant forms of IL-3Rα. Median fluorescence
intensity (MFI) of each antibody was determined for WT IL-3Rα
and for each IL-3Rα mutant. The MFI of WT IL-3Rα
staining for each antibody was set as 100% then the MFI of
each mutant was normalized to a percent of WT staining.

| IL-3Rα construct | 7G3 | CSL362 | 9F5 | 107D2 |
|---|---|---|---|---|
| untransfected | 1.1 | 2.4 | 1.2 | 1.6 |
| WT | 100 | 100 | 100 | 100 |
| E51Q/S59A | 1.4 | 2.4 | 87.0 | 78.1 |
| E51Q/P88T | 1.4 | 2.5 | 70.1 | 35.1 |
| S59A/P88T | 3.0 | 5.0 | 95.6 | 114.5 |
| E51Q/S59A/P88T | 1.0 | 2.9 | 32.7 | 36.3 |

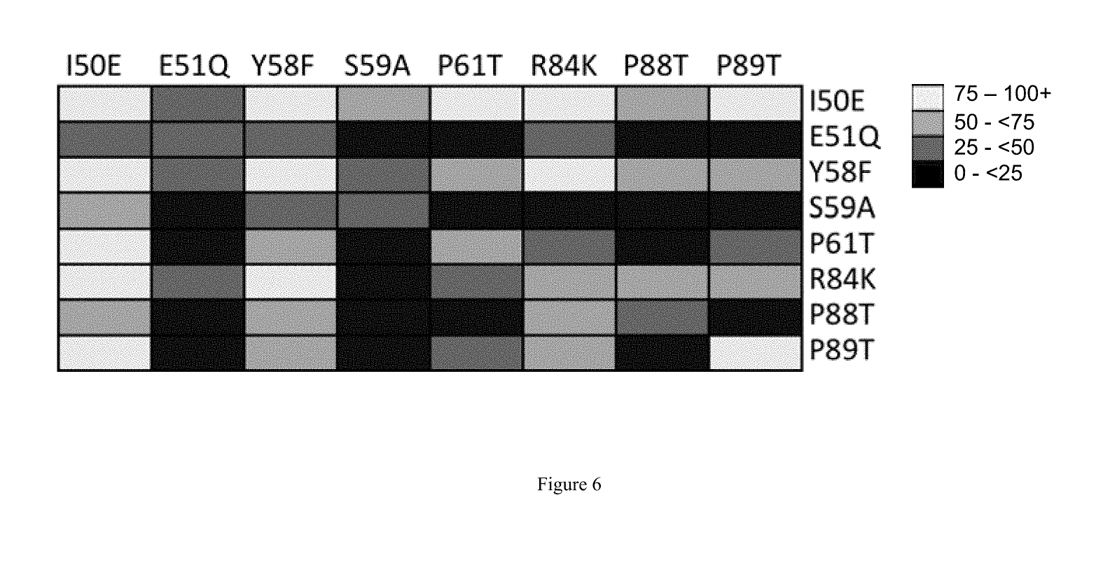

Similar assays were performed to determine the effect of single mutations or combinations of two mutations at positions 50, 51, 58, 59, 61, 84, 88 and/or 89 affected binding of antibody 7G3. In this case, amino acid substitutions were generated and the resulting proteins were expressed in 293FS cells. Binding of anti-hIL-3Rα antibodies to the mutated receptors was determined by flow cytometry and was normalized to binding to the WT hIL-3Rα. Levels of binding of 7G3 was then expressed as a percentage of that observed with 9F5 and is represented as a heatmap in FIG. 6. These data indicate that residues 51, 58, 59, 61, 84, 88 and 89 are involved in binding of 7G3 (and derivatives thereof, such as CSL362) to IL-3Rα. The positions of these residues are also depicted in FIG. 7.

Assays were also performed to determine whether or not residues 51, 59 and/or 88 were required for IL-3 binding and signaling. 293F cells expressing the double or triple combination point mutants of IL-3Rα as described above and CD131 were produced and stimulated with IL-3 (30 ng/ml) for 20 min. STATS activity was measured by flow cytometry after intracellular staining with a phosphoSTAT5 antibody (pSTAT5). Results showed that the mutant forms of IL-3Rα did not substantially reduce or prevent STATS activity compared to wild type IL-3Rα. This effect was also shown to be dose responsive. Using flow cytometry, cells expressing the mutant forms of the receptor were also shown to bind to human IL-3.

Ba/F3 cells expressing CD131 and the double (E51Q/S59A) combination point mutant of IL-3Rα as described above or wild type IL-3Rα were stimulated with an increasing concentration of human IL-3 for 72 h and cell proliferation was determined by tritiated-thymidine incorporation. Results of these assays show that cells expressing mutant IL-3Rα proliferated in response to IL-3. Proliferation of cells expressing wild type IL-3Rα could be blocked with antibody 7G3, whereas proliferation of cells expressing the mutant receptor could not. These data indicate that the residues that reduce or prevent binding of 7G3 to IL-3Rα do not substantially reduce or prevent binding of IL-3 to IL-3Rα.

### Example 2: Mapping a Neutralizing Epitope in IL-3Rα Using Crystallography

A crystal of the Fab of CSL362 and soluble IL-3Rα (sIL-3Rα) produced in insect cells was produced. The resulting crystal contained two sIL-3Rα molecules and two Fab fragments.

Analysis of the crystal showed interactions between the residues/carbohydrates in IL-3Rα and CSL362 shown in Tables 4 and 5. Regions of IL-3Rα that are involved in the interaction with CSL362 are also depicted in FIG. 6.

TABLE 4
residues in V_{L} of CSL362 (SEQ ID NO: 5) that
interact with IL-3Rα (SEQ ID NO: 1).

| CSL362 V_{L} | IL-3Rα |
|---|---|
| Asn31 | Pro61 |
| Gly33 | Val63 |
| Tyr38 | Pro61, |
| Tyr98 | Pro61, Ser59, Met60, |
| Ser99 | Tyr58, Ser59, |
| Tyr100 | Asp57, Tyr58, Ser59, Ala56, |
| Tyr102 | Arg84, Glu51, Ser59 |

TABLE 5
residues in V_{H} of CSL362 (SEQ ID NO: 4) that
interact with IL-3Rα (SEQ ID NO: 1).

| CSL362 V_{H} | IL-3Rα |
|---|---|
| Thr30 | Pro89 |
| Asp31 | Pro89, Asn87, |
| Tyr32 | Pro89 |
| Tyr33 | Pro89, Arg84, Val85, Phe90, Ala86, |
| Asp50 | Arg84 |
| Asn55 | Mannose |
| Gly56 | Mannose |
| Ala57 | N-acetylglucosamine |
| Thr58 | Mannose, Fucose, N-acetylglucosamine |
| Phe59 | Arg84, N-acetylglucosamine, Ser91 |
| Tyr60 | Fucose |
| Gln62 | Ala56 |
| Lys65 | Fucose |
| Leu101 | Ala86, Asn87, Thr48, |
| Leu102 | Ala86, Asp49 |
| Arg103 | Asp49, Ala86, Glu51, |
| Ala104 | Ala86, Arg84, |

Analysis of the crystal structure confirmed the importance of at least residues 51E, 59S, 61P, 84R in IL-3Rα in the interaction with CSL362 (numbering based on SEQ ID NO: 1) as previously determined using mutational analysis. This analysis also indicated that residue 70F of IL-3Rα may be involved in the interaction.

The crystal structure data also indicated that while residue 88 of IL-3Rα may be important for antibody binding, mutations of this residues actually a change in conformation of the receptor thus causing loss of binding. These data indicate that residue 88 of IL-3Rα is not bound by or does not interact with CSL362.

1. A computer-implemented method of organising the marketing of a compound that that binds to interleukin 3 receptor alpha (IL-3Rα) for neutralizing IL-3 signaling, the method comprising:
receiving, over a communications network, information associated with the compound;
processing the information to identify a target market for the compound; and communicating, over the communication network, advertising material to the target market to inform the target market about the compound, wherein the advertising material describes how the compound is used for neutralizing IL-3 signaling by specifically binding to an epitope within IL-3Rα, wherein the epitope is in a region corresponding to amino acids 51-100 of SEQ ID NO: 1.


SEQUENCE LISTING (12 sequences)

SEQ ID NO: 1 - protein, 377 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVAKRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 2 - protein, 120 aa, Mus musculus
EVQLQQSGPELVKPGASVKMSCKASGYTFTDYYMKWVKQSHGKSLEWIGDIIPSNGATFYNQKFKGKATLTVDRSSSTAYMHLNSLTSEDSAVYYCTRSHLLRASWFAYWGQGTLVTVSA

SEQ ID NO: 3 - protein, 114 aa, Mus musculus
DFVMTQSPSSLTVTAGEKVTMSCKSSQSLLNSGNQKNYLTWYLQKPGQPPKLLIYWASTRESGVPDRFTGSGSGTDFTLTISSVQAEDLAVYYCQNDYSYPYTFGGGTKLEIKR

SEQ ID NO: 4 - protein, 120 aa, Artificial [sequence omitted]

SEQ ID NO: 5 - protein, 114 aa, Artificial
  amino acid sequence of light chain variable region of humanized anti-IL-3Ralpha chain antibody CSL362 and modified forms thereof
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKR

SEQ ID NO: 6 - protein, 5 aa, Artificial
  amino acid sequence of HCDR1 of humanized anti-IL-3Ralpha chain antibody CSL362 and modified forms thereof
DYYMK

SEQ ID NO: 7 - protein, 17 aa, Artificial
  amino acid sequence of HCDR2 of humanized anti-IL-3Ralpha chain antibody CSL362 and modified forms thereof
DIIPSNGATFYNQKFKG

SEQ ID NO: 8 - protein, 11 aa, Artificial
  mino acid sequence of HCDR3 of a humanized anti-IL-3R alpha chain antibody
SHLLRASWFAY

SEQ ID NO: 9 - protein, 17 aa, Artificial
  amino acid sequence of LCDR1 of humanized anti-IL-3Ralpha chain antibody CSL362 and modified forms thereof
ESSQSLLNSGNQKNYLT

SEQ ID NO: 10 - protein, 7 aa, Artificial
  amino acid sequence of LCDR2 of humanized anti-IL-3Ralpha chain antibody CSL362 and modified forms thereof
WASTRES

SEQ ID NO: 11 - protein, 9 aa, Artificial
  amino acid sequence of LCDR3 of humanized anti-IL-3Ralpha chain antibody CSL362 and modified forms thereof
QNDYSYPYT

SEQ ID NO: 12 - protein, 881 aa, Homo sapiens
WERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC